SEQUENCE LISTING (9 sequences)

SEQ ID NO: 1 - protein, 967 aa, Rumniococcus flavefaciens
MIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 2 - protein, 7 aa, artificial sequence
  SV40 NLS
PKKKRKV

SEQ ID NO: 3 - protein, 8 aa, artificial sequence
  NLS consensus sequence
PAAKKKLD

SEQ ID NO: 4 - protein, 11 aa, artificial sequence
  HIV NES
LQLPPLERLTL

SEQ ID NO: 5 - protein, 10 aa, artificial sequence
  SV40 NLS extended
PPKKKRKVED

SEQ ID NO: 6 - protein, 9 aa, artificial sequence
  c-myc NLS
PAAKRVKLD

SEQ ID NO: 7 - protein, 16 aa, artificial sequence
  nucleoplasmin NLS
KRPAATKKAGQAKKKK

SEQ ID NO: 8 - protein, 72 aa, Murid gammaherpesvirus 4
GCCAGGGTAGCTCAATTGGTAGAGCATCAGGCTAGTATCCTGTCGGTTCCGGTTCAAGTCCGGGCCCTGGTT

SEQ ID NO: 9 - protein, 84 aa, Hepatitis delta virus
GGCCGGCATGGTCCCAGCCTCCTCGCTGGCGCCGGCTGGGCAACATTCCGAGGGGACCGTCCCCTCGGTAATGGCGAATGGGAC

(19) United States
(12) Patent Application Publication
(10) Pub. No.: US 2024/0041990 A1
(43) Pub. Date: Feb. 8, 2024

(54) APPLICATION OF CRISPR/CAS13 FOR THERAPY OF RNA VIRUS AND/OR BACTERIUM INDUCED DISEASES

(71) Applicant: Helmholtz Zentrum München - Deutsches Forschungszentrum für Gesundheit und Umwelt (GmbH), Neuherberg (DE)

(72) Inventors: Christoph GRUBER, Freising (DE); Dong-Jiunn Jeffery TRUONG, Freising (DE); Florian GIESERT, Muenchen (DE); Wolfgang WURST, Muenchen (DE)

(21) Appl. No.: 18/268,948

(22) PCT Filed: Dec. 21, 2021
(86) PCT No.: PCT/EP2021/086992

§ 371 Date: Jun. 21, 2023

(30) Foreign Application Priority Data
Dec. 22, 2020 (LU) LU102326

(51) Int. Cl. A61K 38/46; C12N 15/11; C12N 9/22; A61K 31/7088; A61P 31/14

(52) U.S. Cl. CPC A61K 38/465; C12N 15/11; C12N 9/22; A61K 31/7088; A61P 31/14; C12N 2310/20; C12N 2310/3513; C12N 2310/335; C12N 2310/321; C12N 2320/32

(57) ABSTRACT

The present invention relates to composition comprising a clustered, regularly interspaced, short palindromic repeats (CRISPR) system comprising i) at least one CRISPR-associated protein 13 (Cas13) or a nucleotide sequence encoding said Cas13 protein fused with at least one nuclear localization signal (NLS) fused to at least one nuclear export sequence (NES) and ii) at least one guide RNA (gRNA) or a nucleotide sequence encoding said gRNA capable of hybridizing with one or more target RNA molecules. Further, the invention relates to a composition comprising a clustered, regularly interspaced, short palindromic repeats (CRISPR) system comprising i) at least one CRISPR-associated protein 13 (Cas13) or a nucleotide sequence encoding said Cas13 protein fused with at least one nuclear localization signal (NLS) or with at least one nuclear export sequence (NES) and ii) at least one guide RNA (gRNA) or a nucleotide sequence encoding said gRNA capable of hybridizing with one or more target RNA molecules, which is fused to at least one viral export element. The present invention also relates to said compositions for use in therapy. In particular, the present invention relates to said compositions for use in a method of preventing or treating a viral or a bacterial disease in a subject. The present invention further relates to nucleic acid molecules comprising a nucleotide sequence encoding said Cas13 protein and/or said gRNA of said compositions, vectors comprising said nucleic acid molecules and host cells comprising the vectors or the nucleic acid molecules. Further, the present invention relates to kits comprising the compositions. The invention also comprises methods of producing said compositions of the present invention.

### CROSS REFERENCE TO RELATED APPLICATION

This application claims priority to LU 102326, filed Dec. 22, 2020. This application is incorporated herein by reference in its entirety.

### FIELD OF THE INVENTION

The present invention relates to composition comprising a clustered, regularly interspaced, short palindromic repeats (CRISPR) system comprising i) at least one CRISPR-associated protein 13 (Cas13) or a nucleotide sequence encoding said Cas13 protein fused with at least one nuclear localization signal (NLS) fused to at least one nuclear export sequence (NES) and ii) at least one guide RNA (gRNA) or a nucleotide sequence encoding said gRNA capable of hybridizing with one or more target RNA molecules. Further, the invention relates to a composition comprising a clustered, regularly interspaced, short palindromic repeats (CRISPR) system comprising i) at least one CRISPR-associated protein 13 (Cas13) or a nucleotide sequence encoding said Cas13 protein fused with at least one nuclear localization signal (NLS) or with at least one nuclear export sequence (NES) and ii) at least one guide RNA (gRNA) or a nucleotide sequence encoding said gRNA capable of hybridizing with one or more target RNA molecules, which is fused to at least one viral export element. The present invention also relates to said compositions for use in therapy. In particular, the present invention relates to said compositions for use in a method of preventing or treating a viral or a bacterial disease in a subject. The present invention further relates to nucleic acid molecules comprising a nucleotide sequence encoding said Cas13 protein and/or said gRNA of said compositions, vectors comprising said nucleic acid molecules and host cells comprising the vectors or the nucleic acid molecules. Further, the present invention relates to kits comprising the compositions. The invention also comprises methods of producing said compositions of the present invention.

### BACKGROUND OF THE INVENTION

From a clinical point of view, the problem with the rapid spread of virus or bacterial strains in general is that the development of a drug takes too much time to be able to develop a therapeutic agent in a reasonable time. Especially with regard to the novel coronavirus SARS-CoV-2 which is a (+)-RNA virus of the Coronaviridae family and which as of early October 2020 has caused over 1,000,000 deaths worldwide, drug development is a critical issue. RNA viruses are also responsible for the other two epi- and pandemics of the recent past (SARS-CoV-1 and MERS-CoV). These two epidemics have in common with COVID-19 to have a high virulence combined with an efficient route of spreading via droplet infections (P. Anfinrud et al. (2020), N Engl J Med 382, 2061-2063; J. Chen (2020) Microbes Infect 22, 69-71).

For this reason, the only means available is the “repurposing” of drugs that have already been approved. For example, Remdesivir, which has originally been developed as a drug for Ebola, is currently being discussed as a therapy for COVID-19 (J. Grein et al. (2020), N Engl J Med 382, 2327-2336). However, since antibodies and small molecule-based therapies such as Remdesivir use the tertiary structure of a protein as the target structure, it is often not possible to apply an approved inhibitor to a new virus or bacteria strain and in addition, viral or bacterial mutations can also change the binding properties of a therapeutic agent.

The prokaryotic immune system CRISPR/Cas acts differently than the immune system of higher eukaryotes. Instead of binding to protein antigens, the CRISPR/Cas systems directly recognize the genetic information of a phage at the ribonucleic acid level. By simply expressing a guide RNA (gRNA) that is complementary to the phage genome, an effector nuclease is directed to the genome of the phage and cutting of the genome is induced (F. Hille et al. (2018), Cell 172, 1239-1259). CRISPR/Cas systems are divided into two classes with six types (K. S. Makarova et al. (2020), Nat Rev Microbiol 18, 67-83). In addition to Cas9, as a programmable DNAse, Cas13 was recently discovered. Unlike Cas9, Cas13 does not cut the DNA but rather the RNA of a phage that attacks the prokaryotic host (O. O. Abudayyeh et al. (2016), Science 353). The nuclease of the Cas13 effector is a HEPN (higher eukaryotes and prokaryotes nucleotide-binding) domain that is split and therefore inactive within the protein. As soon as a target RNA is bound, there is a change in the tertiary structure of the protein, whereby the separated nuclease domain is brought into proximity and activated (O. O. Abudayyeh et al. (2016)).

In the prior art different approaches with Cas13 have already been demonstrated for some model viruses such as influenza A and in an artificial experimental system transfecting synthetic SARS-CoV-2 sequences into human cells expressing already Cas13 (C. A. Freije et al. (2019), Mol Cell 76, 826-837, Abbott et al. (2020), Cell 181, 865-876). Despite the current attempts of developing vaccines against a human-pathogenic virus and also bacterial infection, there remains a need to provide alternative or improved agents, compositions and methods for treatment and/or prevention of diseases caused by human-pathogenic viruses or bacteria. The technical problem underlying the present application is thus to comply with this need.

The technical problem is solved by providing the embodiments reflected in the claims, described in the description and illustrated in the examples and figures that follow.

### SUMMARY OF THE INVENTION

The present inventors have developed a novel CRISPR-Cas-based approach for the treatment of viral diseases, preferably caused by RNA viruses, such as COVID-19, and of bacterial diseases in which the invading or cell replicated virus/bacterial mRNA is bound by so-called Cas13 proteins in the presence of a complementary gRNA (CRISPR-associated RNA) and degraded by the RNAse activity of the Cas13 and thus rendered harmless. Said novel CRISPR/Cas concept targets directly ribonucleic acid sequences instead of the tertiary structure of proteins and can be used as an antiviral/antibacterial therapy that directly attacks the viral RNA sequences particularly of SARS-CoV-2 and/or the bacterial RNA sequences. Such approach is therefore modular and adaptable. The advantage of such an RNA-based therapeutic approach is that the virus genome and also the bacterial genome can be used as a target, which means much easier and faster access to potential therapeutics than addressing the tertiary structure of proteins.

In particular, the present inventors were able to test numerous modifications to the gRNA and the Cas13 protein and identified three components that increased the knockdown efficiency of the Cas13 protein (see FIG. 6, FIGS. 11 and 14). The most important optimization step involved the fusion of at least one nuclear localization signal (NLS) which is also fused to at least one nuclear export signal (NES) to the Cas13 protein (see FIG. 1). Further, it included an extension of the gRNA (see FIGS. 1 and 2) and the fusion of a tRNA to support the folding of the gRNA as well as the fusion of a ribozyme to said gRNA (see FIG. 3). The composition of the present invention comprising all three modifications may refer to the Cas13-IDG as defined elsewhere herein. Additionally, it was found that other modifications of the Cas13 protein and/or the gRNA on the RNA level comprising any one of the following modifications such as modified 5′ and/or 3′ UTRs, post- or cotranscriptional addition of a 5′ CAP structure, replacement of UTP by N1-methylpseudo-UTP, or replacement of the two 5′ and/or 3′ terminal nucleotides by 2′-O-methyl-3′P-thioat, improved knockdown-efficiency of the Cas13 protein even more (see FIGS. 10 and 15). Further, the inventors have also found that another modification of the gRNA on the DNA level which comprises fusing at least one viral export element, preferably fusing at least one constitutive transport element (CTE) or VARdm, most preferably fusing at least one CTE with gRNA increased the knockdown efficiency of the Cas13 protein for DNA delivery, which is fused to at least one NLS which is fused to at least one NES as defined above, even more (see FIG. 16).

The gRNAs are transcribed in the cell nucleus, and according to the inventors, in order to activate the Cas13d RNAse activity, it is first necessary to bind the gRNA (see FIG. 4). Ideally, this step can only take place in the cell nucleus. In contrast, the viral or bacterial mRNA target molecules after viral or bacterial infection are essentially located in the cytosol. The fusion of said localization signals led to the localization of Cas13 protein into the cellular cytoplasm and to a stabilization of the protein in this compartment. In contrast to the already published Cas13 variant, this step for the first time allows to degrade cytosolic instead of nuclear RNAs. This step is essential, since the replication of for example numerous RNA viruses, such as coronaviruses, takes place exclusively in the cytosol. However, if the composition of the present invention is not delivered as a DNA based system as mentioned above, but as a RNA or protein based system, said novel composition comprising the above mentioned modification(s) is also of high importance e.g. for viruses that are located in the nucleus and in the cytosol, such as for the family of Orthomyxoviridae, preferably an influenza virus, where again a balanced localization of Cas13 protein is needed in both cell compartments which is achieved by the fusion of Cas13 protein to at least one NLS fused to at least one NES.

In sum, this highly unconventional and non-obvious approach has proven to be the most suitable of several tested strategies for directing translated Cas13 protein into the nucleus for loading with gRNA and then exporting it into the cytosol or vice versa as mentioned above, thereby leading to an increase in efficiency in the degradation of the desired target structures—thus having tremendous potential in diagnosis and therapeutics (see FIGS. 6 and 7).

Accordingly, in a first aspect, the present invention relates to a composition comprising a clustered, regularly interspaced, short palindromic repeats (CRISPR) system comprising i) at least one CRISPR-associated protein 13 (Cas13) or a nucleotide sequence encoding said Cas13 protein fused with at least one nuclear localization signal (NLS) fused to at least one nuclear export sequence (NES) and ii) at least one guide RNA (gRNA) or a nucleotide sequence encoding said gRNA capable of hybridizing with one or more target RNA molecules.

The present invention may also disclose the composition as defined elsewhere herein, wherein said Cas13 protein is fused with said at least one NLS fused to said at least one NES via a linker.

The inventors have now also found out that the same effect, namely an increased efficiency in the degradation of the desired target structures, is achieved by a composition comprising a clustered, regularly interspaced, short palindromic repeats (CRISPR) system comprising i) at least one CRISPR-associated protein 13 (Cas13) or a nucleotide sequence encoding said Cas13 protein fused with at least one nuclear localization signal (NLS) or with at least one nuclear export sequence (NES) and ii) at least one guide RNA (gRNA) or a nucleotide sequence encoding said gRNA capable of hybridizing with one or more target RNA molecules, which is fused to at least one viral export element. Here, by fusing at least one viral export element, which is preferably a constitutive transport element (CTE) or a minihelix of adenovirus VA1 RNA (VARdm), most preferably CTE to said gRNA, cytosolic knockdown by Cas13-NES or NLS is increased (see FIG. 17).

Encompassed herein is also the compositions as defined elsewhere herein, wherein said Cas13 protein is a Cas13d protein.

Also described herein is the compositions as defined elsewhere herein, wherein said Cas13d protein is derived from the genus of Ruminococcus, preferably from Ruminococcus flavevaciens.

The present invention may also disclose the compositions as defined elsewhere herein, wherein said gRNA has a length of at least about 23 nucleotides.

The present invention may also disclose the compositions as defined elsewhere herein, wherein said gRNA has a length of between about 26 to about 30 nucleotides.

Also described herein is the compositions as defined elsewhere herein, wherein said gRNA has at least about 80% complementary sequence identity to said one or more target RNA molecules.

Encompassed herein is also the compositions as defined elsewhere herein, wherein said gRNA is capable of hybridizing to (a) 5′- and/or 3′-untranslated region(s) of said one or more target RNA molecules.

The present invention may also disclose the compositions as defined elsewhere herein, wherein said nucleotide sequence encoding said gRNA is fused with a tRNA or a ribozym.

The present invention may also disclose the compositions as defined elsewhere herein, wherein said one or more target RNA molecules are viral or bacterial target RNA molecules.

The present invention may also disclose the compositions as defined elsewhere herein, wherein said compositions are for inactivating bacterial or viral ssRNA.

Encompassed herein is also the compositions as defined elsewhere herein, wherein said compositions are pharmaceutical compositions, preferably further comprising at least one pharmaceutical acceptable carrier.

In a second aspect, the present invention also relates to the compositions for use in therapy.

In a third aspect, the present invention further relates to the compositions for use in a method of preventing or treating a viral or bacterial disease in a subject, the method comprising administering to the subject a therapeutically effective amount of the said compositions.

Further, the present invention may also disclose the compositions for the use as defined elsewhere herein, wherein the viral disease is caused by a RNA virus, preferably the viral disease is any one of a coronavirus disease, influenza A, ebola, measles, hepatitis C, tick-borne encephalitis (TBE), Venezuelan Equine Encephalitis (VEE) viral infection, dengue fever, yellow fever, or zika fever, even more preferably the viral disease is the COVID-19 disease.

In a fourth aspect, the present invention relates to nucleic acid molecules comprising a nucleotide sequence encoding said Cas13 protein and said gRNA as defined elsewhere herein, vectors comprising said nucleic acid molecules as defined herein or to host cells comprising said nucleic acid molecules or said vectors as defined elsewhere herein.

In a fifth aspect, the present invention also relates to kits comprising said compositions as defined elsewhere herein. The present invention may also disclose the kits as defined elsewhere herein, further comprising a delivery system and/or a label.

In a sixth aspect, the invention also relates to methods of producing the compositions of the present invention, wherein the compositions are produced starting from the nucleic acid coding said Cas13 protein and said gRNA as defined herein by means of genetic engineering methods, thereby producing said compositions; optionally obtaining said produced compositions.

### BRIEF DESCRIPTION OF THE FIGURES

FIG. 1: Knockdown efficiency of different Cas13 protein und gRNA variants. Each variant was targeted against a co-transfected firefly luciferase and normalized to a non-target gRNA. The fusion of a NLS-NES tandem signal to Cas13d improved the efficiency compared to the NLS-only variant. Elongation of the gRNA further improved the efficiency. All conditions were compared to the latest generation of miRNAs/RNA interference (Fellmann et al., 2013 in Cell reports).

FIG. 2: Characterization of different gRNA lengths for Cas13d-NLS-NES for targeting a co-transfected nanoluciferase. The optimal length of the gRNA is in the range of 26 bp 30 bp.

FIG. 3: Fusion of either a tRNA (MHV68 M1-7) or a ribozyme (HDV) to a 30 bp gRNA increases the knockdown efficiency against a co-transfected nanoluciferase for Cas13d-NLS-NES.

FIG. 4: Different strategies to export the gRNA from the nucleus to the cytosol are compared. Knockdown efficiencies against a co-transfected nanoluciferase were measured for different Cas13d protein localizations and different gRNA expression strategies. Polymerase III driven gRNAs remain in the nucleus. Therefore, nuclear localized Cas13d (NLS) is superior to cytosolic Cas13d (NES). A tandem fusion (NLS-NES) maximizes the knockdown efficiency, because of picking up the gRNA in the nucleus and being active against the cytosolic mRNA coding for Nanoluciferase. Western Blot confirms that a gRNA being present in the same compartment as Cas13d, stabilizes the protein. A complementary strategy to export the gRNA by expression from a Polymerase II driven promoter does not improve the efficiency.

FIG. 5: Knockdown efficiency of different Cas13d protein und gRNA variants. Each variant was targeted against a co-transfected firefly luciferase and normalized to a non-target gRNA. A co-transfected nanoluciferase, measured in the same well, but not directly targeted by the gRNA, is the read-out for target RNA induced collateral activity. The fusion of a NLS-NES tandem signal improved the efficiency compared to the NLS-only variant. Elongation of the gRNA further improved the efficiency. All conditions were compared to the latest generation miRNA/RNA interference.

FIG. 6: (A) Schematic representation of the assay for the development of an optimized Cas13 system. (B) Comparison of different Cas13 systems to enhanced Cas13-IDG (NLS-NES, 30 bp gRNA, 3′tRNA). Knockdown efficiency was measured by a co-transfected nanoluciferase. (C) Partially cytosolic localization of Cas13d-NLS-NES compared to Cas13d-NLS. (D) Expected mechanism of Cas13d's impact on the cellular translation through direct degradation of the coding mRNA and secondary cutting of a specific position of the 28S rRNA. (E) Cas13d-NLS-NES targeted against a co-transfected mRuby3 and subsequent analysis of the ribosomal RNA on a bioanalyzer chip.

FIG. 7: (A) Anticipated mode of action of an antiviral strategy based on Cas13. Four level viral inhibition is achieved by targeting the viral genome directly, related genome copies, subgenomic mRNAs or inhibiting the viral protein translation. (B) Efficient knockdown of the artificially expressed viral protein RNA-dependent RNA polymerase (RdRp) (HA tagged), confirmed via Western Blot. In addition, the collateral effect was confirmed by co-transfecting an unrelated mRuby3 protein. (C) HEK293T cells, transfected with ACE2, Cas13d-NLS-NES and gRNAs were infected with SARS-CoV-2 and 72 h later the viral load was quantified via RT-qPCR.

FIG. 8: The global cellular protein translation was measured for cells transfected with either active or inactive Cas13d-NLS-NES, a gRNA and a target RNA.

FIG. 9: Agarose gel of extracted RNA form cells transfected either with or without Cas13d-NLS-NES, a target RNA and a complementary gRNA. The 28S rRNA cleavage products is indicated. The same 28S rRNA cleavage product is found in vitro for purified 80S ribosomes incubated either with or without purified Cas13d, a gRNA and a target RNA.

FIG. 10: (A) Improved knockdown-efficiency for RNA delivery of Cas13 by optimizing different aspects of the transfected Cas13 mRNA or gRNA. Efficiency was measured by targeting a co-transfected nanoluciferase and normalized to a non-target gRNA. (B) Expression levels of Cas13 from mRNA were improved by modifying UTRs, the 5′ CAP structure or the replacement of UTP by N1-Methylpseudo-UTP.

FIG. 11: (A) Illustration of Cas13d variants, fused to different localization signals. (B) Knockdown efficiency of differentially localized Cas13d variants against co-transfected nanoluciferase target RNA (crRNA 1 and crRNA 2). The balanced set of nuclear and cytosolic localization signals of Cas13d-IDG maximises the knockdown efficiency for two crRNAs.

FIG. 12: (A) Representative images showing the localization of Cas13d protein variants, fused to different localization signals. (B) Quantification of cytosolic/nuclear distribution of different Cas13d variants for 100 cells.

FIG. 13: Stability of Cas13d variants fused to different localization signals, analysed by Western Blot, for a polymerase II driven gRNA (cytosolic) and a polymerase III driven gRNA (nuclear). Protein/gRNA complexed are most stable in conditions where both components are localized to the same compartment.

FIG. 14: Comparison of knockdown efficiencies against Venezuelan Equine Encephalitis (VEE) replicon for semi-cytosolic Cas13-NLS-NES and nuclear localized Cas13-NLS for gRNAs targeting different structural elements, measured by reduction of mGreenLantern expression. Self-replicating VEE RNA consists of viral untranslated regions (UTR), non-structural protein (nsp), interferon-inhibiting proteins (B18R, E3L), mGreenLantern (mGL) reporter and puromycin resistance gene. Knockdown of cytosolic replicating VEE is strongly enhanced for Cas13d-NLS-NES.

FIG. 15: (A) Continuous analysis of SARS-CoV-2-GFP replication for treatment conditions with Cas13d-IDG and different gRNAs, targeting structural viral elements (NT: non-target, CDS: coding sequences, UTR: untranslated regions). A chemically modified 3′UTR gRNA is most efficient in inhibiting viral replication. (B) Targeting of different SARS-CoV-2 variants by Cas13d-IDG. Knockdown efficiency was analysed by RT-qPCR for two viral target genes in biological hexaplicates and technical duplicates.

FIG. 16: Comparison of knockdown efficiencies for two gRNAs targeting a co-transfected nanoluciferase for NES or tandem NLS-NES fused to Cas13. Additionally, either an unmodified, or CTE fused gRNA was tested. The combination of NLS-NES and CTE-gRNA maximizes the knockdown efficiency.

FIG. 17: (A) Illustration of different crRNA export strategies. Polymerase III (pol III) driven crRNAs remain in the nucleus, polymerase II (pol II) driven crRNA are exported to the cytosol, crRNAs fused to are constitutive transport element (CTE) are exported, as well as crRNAs fused to a minihelix of adenovirus VA1 RNA (VARdm). (B) Comparison of knockdown efficiencies against nanoluciferase target RNA (crRNA 1 and crRNA 2) for different crRNA export motifs together with either cytosolic (NES) or nuclear (NLS) Cas13d protein. CrRNAs fused to a CTE export motif is most efficient in driving Cas13d based knockdown in the cytosol.

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments described throughout the specification should not be construed to limit the present invention to only the explicitly described embodiments. This description should be understood to support and encompass embodiments which combine the explicitly described embodiments with any number of the disclosed and/or preferred elements. Furthermore, any permutations and combinations of all elements described herein should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e. the subject-matter consists in the inclusion of a stated member, integer or step or group of members, integers or steps. When used herein the term “comprising” can be substituted with the term “containing” or “including” or sometimes when used herein with the term “having”. When used herein “consisting of” excludes any element, step, or ingredient not specified.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Unless otherwise indicated, the term “at least” preceding a series of elements is to be understood to refer to every element in the series. The term “at least one” refers to one, two, three or more such as four, five, six, seven, eight, nine, ten and more. Those skilled in the art will recognize, or be able to ascertain using no more than routine experimentation, many equivalents to the specific embodiments of the invention described herein. Such equivalents are intended to be encompassed by the present invention.

The term “less than” or in turn “more than” or “below” does not include the concrete number.

The term “and/or” wherever used herein includes the meaning of “and”, “or” and “all or any other combination of the elements connected by said term”.

When used herein “consisting of” excludes any element, step, or ingredient not specified in the claim element. When used herein, “consisting essentially of” does not exclude materials or steps that do not materially affect the basic and novel characteristics of the claim.

The term “including” means “including but not limited to”. “Including” and “including but not limited to” are used interchangeably.

The term “about” means plus or minus 20%, preferably plus or minus 10%, more preferably plus or minus 5%, most preferably plus or minus 1%.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be gained from the examples, offered for illustrative purposes only. The examples are not intended to limit the scope of the present invention in any way.

### CRISPR Composition

### Cas13 Protein

The present invention refers to a composition comprising a CRISPR system as described elsewhere herein. Such CRISPR-Cas system stands for “clustered, regularly interspaced, short palindromic repeats (CRISPR)/CRISPR-associated protein”. It is based on an adaptive defense mechanism evolved by bacteria and archaea to protect them from invading viruses, bacteria and plasmids, which relies on small RNAs for sequence-specific detection and silencing of foreign ribonucleic acids. CRISPR/Cas systems are composed of cas genes organized in operon(s) and CRISPR array(s) consisting of genome-targeting sequences (called spacers) interspersed with identical repeats (Bhaya et al. (2011) Annu Rev Genet 45:273-297; Barrangou R and Horvath P (2012) Annu Rev Food Sci Technol 3:143-162). Target recognition by guide RNAs (gRNAs) directs the silencing of the foreign sequences by means of Cas proteins that function in complex with the gRNAs.

In more detail and in general, a CRISPR-Cas or CRISPR system refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system).

In the present invention said composition comprising said particular system refers to a CRISPR-Cas13 system. In the present invention the composition comprising said CRISPR system comprises at least one (one or more) clustered, regularly interspaced, short palindromic repeats (CRISPR)-associated (Cas) protein, which is a Cas13 protein. Thus, the composition comprises at least one (one or more) such as one, two, three, four, five or more Cas13 proteins as defined elsewhere herein. CRISPR-Cas13 is an RNA targeting and editing system based on the bacterial immune system that protects them from viruses and bacteria. Cas13 is the effector protein that targets and cleaves invading ribonucleic acids from viruses and bacteria in type VI CRISPR-Cas systems. The CRISPR-Cas13 system is analogous to the CRISPR-Cas9 system. However, unlike Cas9 which targets DNA, Cas13, a Rnases, targets/detects and cleaves/degrades single stranded RNA (ssRNA). Thus, a Cas13 protein also refers to a (Type VI) RNA-targeting effector protein. Cas13 enzymes have two higher eukaryotes and prokaryotes nucleotide-binding (HEPN) endoRNase domains that mediate precise RNA cleavage with a preference for targets with protospacer flanking sites (PFSs) observed biochemically and in bacteria. Example RNA-targeting effector proteins include C2c2 (now known as Cas13a), Cas13b, Cas13c and Cas13d. Cas13 was first discovered in L. shahii, a species of the Leptotrichia bacteria while researchers were looking for previously unidentified CRISPR systems. Since Cas13 proteins were identified in 2016 by the research group of Feng Zhang (Broad Institute, MIT), four different subtypes (Cas13a-d) have been described and intensively studied so far. They differ strongly in their sequences, but have as a common feature so-called two HEPN domains (Higher Eukaryotes and Prokaryotes Nucleotide Binding Domains), which are responsible for RNAse activity. Such domains of said Cas13 protein mediate precise RNA cleavage with a preference for targets with protospacer flanking sites (PFSs). In contrast, Cas13 proteins do not show any activity against DNA as Cas9 proteins as previously mentioned.

In one embodiment, the Cas13 protein comprises at least one HEPN domain, including but not limited to the HEPN domains known in the art, and domains recognized to be HEPN domains by comparison to consensus sequence motifs. In certain embodiments, the Cas13 protein comprises a single HEPN domain. In certain other embodiments, the Cas13 protein comprises two HEPN domains. In another embodiment, the Cas13 protein comprises one or more HEPN domains comprising a RxxxxH motif sequence. The RxxxxH motif sequence can be, without limitation, from a HEPN domain known in the art. RxxxxH motif sequences further include motif sequences created by combining portions of two or more HEPN domains. As noted, consensus sequences can be derived from the sequences of the orthologs disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application entitled “Novel Type VI CRISPR Orthologs and Systems,” labeled as attorney docket number 47627-05-2133 and filed on Apr. 12, 2017.

Since said composition of the present invention targets RNA molecules, wherein the at least one Cas13 protein forms a complex with the at least one gRNA and wherein the at least one gRNA directs the complex to the one or more target RNA molecules, thereby targeting the one or more target RNA molecules, said composition comprising said CRISPR system may also refer to a composition comprising a ribonucleic acid detection system. Since said composition not only targets RNA molecules via said Cas13 Rnase, but then also cleaves said target RNA molecules, thereby degrading said RNA molecules, said composition comprising said CRISPR system may also refer to a composition comprising a ribonucleic acid degradation system.

In one embodiment, the at least one Cas13 protein or the nucleotide sequence encoding said protein is Cas13a protein or a functional variant thereof or a homologue or an orthologue thereof. In another embodiment, the at least one Cas13 protein or the nucleotide sequence encoding said protein is Cas13b protein or a functional variant thereof or a homologue or an orthologue thereof. In an even another embodiment, the at least one Cas13 protein or the nucleotide sequence encoding said protein is Cas13c protein or a functional variant thereof or a homologue or an orthologue thereof. The Cas13a protein may be from an organism selected from the group consisting of Leptotrichia, Listeria, Cotynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter and Lachnospira or the Cas13a protein is selected from the group consisting of: Leptotrichia shahii, Leptotrichia wadei, Listeria seeligeri, Lachnospiraceae bacterium, Clostridium aminophilum, Carnobacterium gallinarum, Paludibacter propionicigenes, Listeria weihenstephanensis, Rhodobacter capsulatus, preferably from Leptotrichia wadei. In another embodiment said Cas13a protein may be selected from an amino acid sequence having at least 80% sequence identity to any of the sequences listed in Table 4 and 5 of US20200165594, which is herein incorporated by reference. In certain embodiments, Cas13b is from an organism selected from Bergeyella, Prevotella, Porphyromonas, Bacteroides, Alistipes, Riemerella, Capnocytophaga, Flavobacterium, Myroides, Chryseobacterium, Paludibacter, Psychroflexus, Phaeodactylibacter Sinomicrobium, Reichenbachiella, preferably Prevotella, even more preferably Prevotella sp. P5-125. In another embodiment, the Cas13b protein is, or comprises an amino acid sequence having at least 80% sequence identity to any of the sequences listed in Table 6 of US20200165594, which is herein incorporated by reference. In certain embodiments, the Cas13c protein is one as disclosed in U.S. Provisional Patent Application No. 62/525,165 filed Jun. 26, 2017, and PCT Application No. US 2017/047193 filed Aug. 16, 2017.

In a preferred embodiment of the present invention the Cas13 protein or the nucleotide sequence encoding said protein is Cas13d or a functional variant thereof or a homologue or an orthologue thereof. Cas13d protein may be from an organism of a genus selected from the group consisting of Leptotrichia, Listeria, Colynebacter, Sutterella, Legionella, Treponema, Filifactor, Eubacterium, Ruminococcus, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Campylobacter, and Lachnospira, preferably from Eubacterium or Ruminococcus, most preferably from Ruminococcus. It has been shown that Cas13d from Ruminococcus flavefaciens, preferably from Ruminococcus flavefaciens XPD3002, is most efficient to degrade target RNAs. Thus, in an even more preferred embodiment, the Cas13d protein or the nucleotide sequence encoding said protein is derived from Ruminococcus, most preferably derived from Ruminococcus flavefaciens. By derived, it is meant that the derived protein is largely based, in the sense of having a high degree of sequence homology with, a wildtype protein, here with Cas13d from Ruminococcus, preferably from Ruminococcus flavefaciens, and thus provides the same function as said wildtype protein, but that it has been mutated (modified) in some way as known in the art or as described herein. A Cas13d protein that is derived from Ruminococcus, preferably from Ruminococcus flavefaciens, may also refer to a functional variant as defined elsewhere herein. A composition as defined herein, wherein said Cas13 protein is from the genus of Ruminococcus, preferably from Ruminococcus flavefaciens, is also comprised herein and mostly preferred.

In particular embodiments, the functional variant of said Cas13d protein as defined elsewhere herein has a sequence homology or identity of at least about 50%, or at least about 60%, or at least about 70%, or at least about 80%, more preferably at least about 85%, even more preferably at least about 90%, such as for instance at least about 95%, at least about 96%, at least about 97%, at least about 98% or even at least about 99% amino acid sequence identity with the wildtype amino acid sequence of the Cas13d protein of Ruminococcus flavefaciens (SEQ ID NO. 1). Thus, the present invention also comprises the composition as defined elsewhere herein, wherein the Cas13 protein or the nucleotide sequence encoding said protein is a Cas13d protein comprising an amino acid sequence with at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%,, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO.: 1.

A “functional variant” of a protein as used herein refers to a variant of such protein which retains at least partially the activity of that protein. Functional variants may include mutants (which may be insertion, deletion, or substitutions as known to a person skilled in the art), including polymorphs, etc. Also included within functional variants are fusion products of such protein with another, usually unrelated, nucleic acid, protein, polypeptide or peptide. Functional variants may be naturally occurring or may be man-made. Advantageous embodiments can involve engineered or non-naturally occurring Cas13 proteins as defined herein. Thus, a functional variant of a Cas13 protein as described herein retains the biological activity of the Cas13 protein from which it is derived. Generally, a Cas13 protein variant has at least about 50%, 60%, 70%, 75%, 80%, 85%, 90%, 95%, 96%, 97%, 98% or even at least about 99% amino acid sequence identity with the Cas13 protein from which it is derived.

The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related. In particular embodiments, the homologue or orthologue of a Cas13 protein such as Cas13a, b, c or d as referred to herein has a sequence homology or identity of at least 80%, more preferably at least 85%, even more preferably at least 90%, such as for instance at least 95% with a Cas13 protein such as Cas13a, b, c or d. In further embodiments, the homologue or orthologue of a Cas13 protein such as Cas13a, b, c or d as referred to herein has a sequence identity of at least 80%, more preferably at least 85%, even more preferably at least 90%, such as for instance at least 95% with the wild type Cas13 protein such as Cas13a, b, c or d. Each embodiment which refers to the Cas13 protein as defined herein may also be applicable to the functional variant thereof or a homologue or a orthologue thereof.

By “identity” or “sequence identity” is meant a property of sequences that measures their similarity or relationship. The term “sequence identity” or “identity” as used in the present invention means the percentage of pair-wise identical residues—following (homology) alignment of a sequence of a polypeptide of the invention with a sequence in question—with respect to the number of residues in the longer of these two sequences. Identity is measured by dividing the number of identical residues by the total number of residues and multiplying the product by 100. The percentage of sequence identity can, for example, be determined herein using the program BLASTP, version blastp 2.2.5 (Nov. 16, 2002; cf. Altschul, S. F. et al. (1997) Nucl. Acids Res. 25, 3389-3402). In this embodiment the percentage of homology is based on the alignment of the entire polypeptide sequences (matrix: BLOSUM 62; gap costs: 11.1; cutoff value set to 10-3) including the respective sequences. It is calculated as the percentage of numbers of “positives” (homologous amino acids) indicated as result in the BLASTP program output divided by the total number of amino acids selected by the program for the alignment.

In certain embodiments, a protospacer adjacent motif (PAM) or PAM-like motif directs binding of the Cas13 protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence” as also mentioned elsewhere herein.

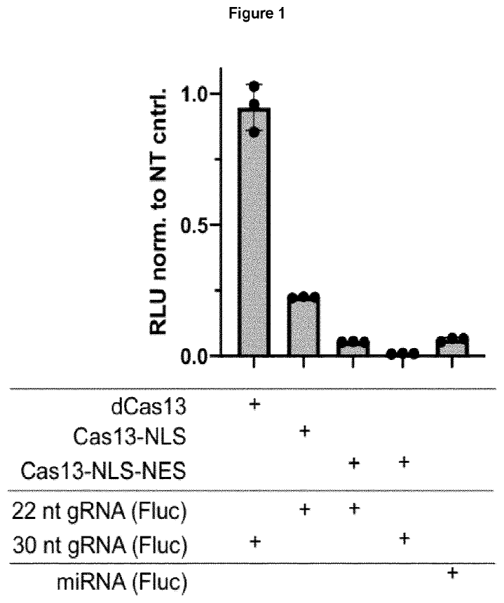

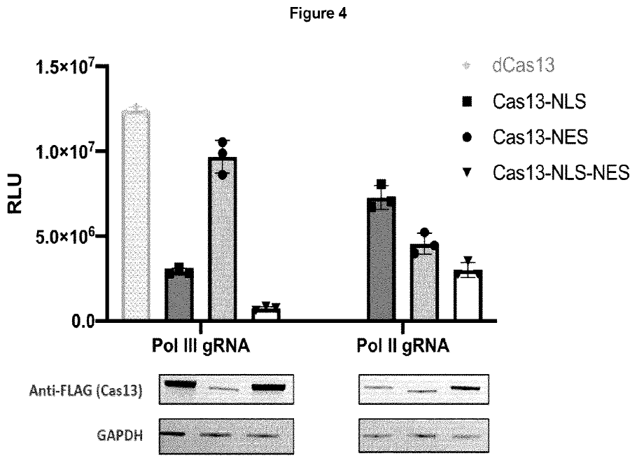

The increased efficiency to degrade target RNA molecules as mentioned elsewhere herein is due to the specific design of the system of the present invention. The inventors were able to show that the cellular localization of Cas13 protein has a decisive effect on the degradation efficiency of the overall composition and requires the presence of Cas13 in both the cell nucleus and the cytosol as previously mentioned. The novel composition comprising said particular CRISPR system uses an inventive tandem combination of NLS (nuclear localisation signal) and NES (nuclear export sequence). On the protein level, a 90% reduction of the virus SARS-CoV-2 RNA-dependent RNA-Polymerase (RdRp) could thus be detected as shown by the Examples (see also FIGS. 1 and 4).

Therefore, the Cas13 protein of the CRISPR system comprised by the composition of the present invention is always fused with at least one (e.g. one, two, three, four, five, six or more) nuclear localization signal (NLS), which is fused to at least one (e.g. one, two, three, four, five, six or more) nuclear export sequence (NES). This modification to said system refers to the most important modification. A NES is a short target peptide containing 4 hydrophobic residues in a protein that targets said protein, namely Cas13 for export from the cell nucleus to the cytoplasm through the nuclear pore complex using nuclear transport, when for example the composition comprising said system is delivered as a DNA based system. A NLS targets a protein, namely Cas 13, located in the cytoplasm for import to the nucleus, when for example the composition comprising said system is delivered as a RNA or protein based system as defined elsewhere herein.

Said at least one NLS fused to said at least one NES refers to a (tandem) localization signal, which thus allows the Cas13 protein to shuttle between the nucleus, where it may get activated by binding to said at least one gRNA, and the cytosol, where the target RNA molecules can be located after for example a viral or bacterial infection of a subject. Viruses that also enter the nucleus of a cell such viruses from the family Orthomyxoviridae, preferably influenza provide target RNA molecules also in the nucleus as well as in the cytosol, where the composition again needs the localization signal as defined herein, when the composition may be delivered on a RNA or a protein level to allow the system to shuttle from the cytosol to the nucleus to degrade the target RNA molecules in the nucleus as well.

Said Cas13 protein as defined herein is preferably fused to said localization signal (in other words said at least one NLS fused to said at least one NES) via any peptide linker known to a person skilled in the art of at least 1 amino acid in length. Thus, in this context, the wording that said Cas13 protein as defined herein is linked to said at least one NLS fused to said at least one NES may also be used herein. This may also refer to an indirect fusion or linkage, since a peptide linker is applied. Thus, the wording “indirectly fused” or “indirectly linked” can also be used herein in this context. Said linker is preferably 1 to 20 amino acids in length. More preferably, the linker is 1 to 15 amino acids in length, and even more preferably, the linker is 1 to 10 amino acids in length, such as 1 to 5 amino acids in length. Even more preferably, the linker is 10, 9, 8, 7, 6, 5, 5, 3, 2, 1 amino acid(s) in length. It is preferred that the linker molecule is a linear or a helical linker, even more preferably the linker is a helical linker. It is further preferred that the linker is a flexible linker using e.g. the amino acids glycine and/or serine. In a particularly preferred embodiment of the invention, between 50% and 100%, particularly between 60% and 100%, particularly between 70% and 100%, particularly between 80% and 100%, particularly between 90% and 100%, and especially 100% of the amino acid residues of the linker molecule are glycine and serine residues, preferably forming an alpha-helix structure.

The fusion/linkage between the Cas13 protein and the localization signal preferably includes a GlySer linker such as GGGS. They can be used in repeats of 3 ((GGGS)3) or 6, 9 or even 12 or more, to provide suitable lengths, as required. The linkers used to engineer appropriate amounts of “mechanical flexibility”. In a preferred embodiment, said linker is a GlySerThr (GST) linker as defined herein. The linkage of the Cas13 protein to said localization signal may be covalently. The present invention thus further comprises that the Cas13 protein is covalently fused/linked with said at least one NLS fused to said at least one NES as defined herein. The term “covalently linked/fused” in this context and also as used throughout the present invention refers to covalent bonds that are typically formed by the sharing of electron pairs between atoms. In accordance with the present invention and when the term “covalently fused/linked” is used, a covalent bond is formed between said Cas13 protein to said localization signal as defined herein by use of a peptide linker as described above or such covalent bond is formed between said at least one NLS (also among each other, if more than one NLS is used) and then between said at least one NES (also among each other, if more than one NES is used) as defined elsewhere herein.

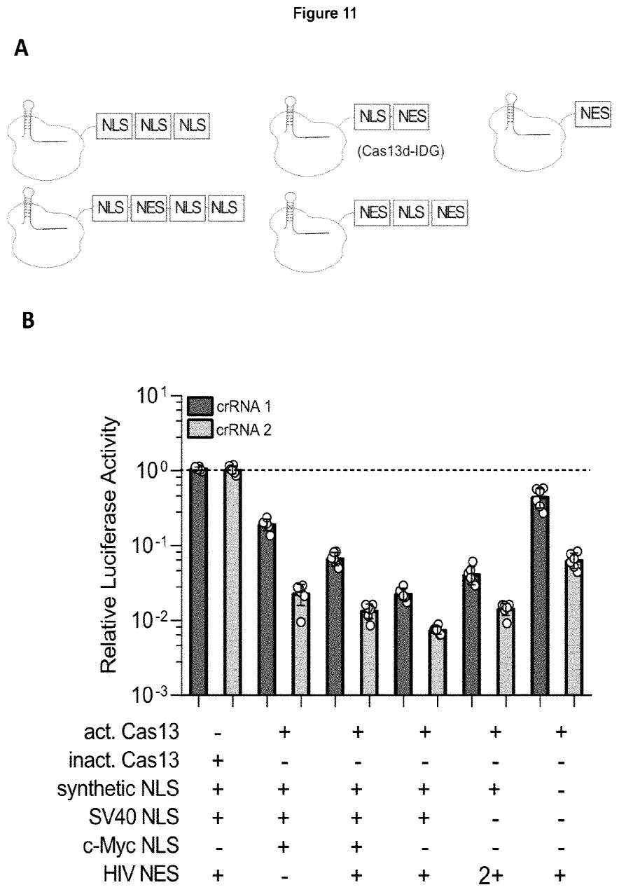

Said localization signal comprising said at least one NLS fused to said at least one NES as defined elsewhere herein may be fused to the N-terminal or C-terminal end of said Cas13 protein. Thus, in one embodiment, said localization signal comprising said at least one NLS fused to said at least one NES as defined elsewhere herein is fused to the C-terminal end of said Cas13 protein. In another embodiment, said localization signal comprising said at least one NLS fused to said at least one NES as defined elsewhere herein is fused to the N-terminal end of said Cas13 protein. In a preferred embodiment, said localization signal comprising said at least one NLS fused to said at least one NES as defined elsewhere herein is fused to the C-terminal end of said Cas13 protein. According to the present invention said at least one NLS of said signal may be fused directly to said at least one NES, meaning that the amino acid sequences (also called peptide sequences) are arranged one after the other without a linker as defined elsewhere herein. A “direct fusion” or “fusing directly” as used throughout the present invention thus refers to fusing said at least one NLS to said at least one NES as defined elsewhere herein without any linker, thereby forming the localization signal comprising at least one NLS and at least one NES. The definition of a direct fusion as above can be applied in any context concerning a direct fusion. The direct fusion or “fusing directly” also refers to the fusion of said NLS or NES among each other, if more than one NLS or NES is used for said localization signal. In this context, any arrangement, no matter if said Cas13 protein is firstly fused to said at least one NLS or firstly fused to said at least one NES of said localization signal is comprised by the present invention. Thus, said Cas13 protein as defined herein may be fused/linked as described firstly to at least one NLS followed by at least one NES or vice versa. Also when one or more NLS and one or more NES are used for said localization signal, any arrangement can be possible in between said localization signal (e.g. NLS-NES-NLS or NES-NES-NLS or NLS-NLS-NES-NES etc.) and is thus comprised by the present invention. Non-limiting examples of NLSs include, but are not limited to a NLS sequence derived from the NLS of the SV40 virus large T-antigen having the amino acid sequence PKKKRKV (SEQ ID NO: 2), a NLS-sequence derived from the extended version of said NLS of the SV40 virus large T-antigen having the amino acid sequence PPKKKRKVED (SEQ ID NO: 5), a NLS consensus sequence (also called synthetic NLS) having the amino acid sequence PAAKKKLD (SEQ ID NO: 3), a c-myc NLS having the amino acid sequence PAAKRVKLD (SEQ ID NO: 6), or a nucleoplasmin bipartite NLS with the amino acid sequence KRPAATKKAGQAKKKK (SEQ ID NO: 7). Non-limiting examples of NESs include, but are not limited to a NES sequence derived from the NES of the HIV virus having the amino acid sequence LQLPPLERLTL (SEQ ID NO: 4). In a preferred embodiment, the at least one NLS fused to said at least one NES as defined herein comprises the SV40 NLS having the amino acid sequence as depicted in SEQ ID NO: 5, the HIV NES having the amino acid sequence as depicted in SEQ ID NO: 4, and the NLS consensus sequence having the amino acid sequence as depicted in SEQ ID NO: 3 (see FIG. 11). In this regard, any arrangement of said at least one NLS (also more than one NLS according to SEQ ID NO: 5 and/or more than one NLS according to SEQ ID NO: 3) as mentioned and of said at least one NES (also more than one NES according to SEQ ID NO: 4) as defined is comprised herein. In an even more preferred embodiment, said SV40 NLS having the amino acid sequence as depicted in SEQ ID NO: 5 is fused/linked to the Cas13 protein, preferably to the C-terminal end of said Cas13 protein, and followed by/fused to said HIV NES having the amino acid sequence as depicted in SEQ ID NO: 4, which is then followed by/linked to said NLS consensus sequence (also called synthetic NLS) having the amino acid sequence as depicted in SEQ ID NO: 3. Thus, the composition of the invention as defined herein preferably comprises two NLS and one NES fused to said Cas13 protein as defined herein, even more preferably wherein the Cas13 protein of the composition as defined herein is fused with two NLS fused to one NES.

In some embodiments, the Cas13 protein as described herein can be fused to one or more peptide tags, including a His-tag, GST-tag, or myc-tag. In some embodiments, the Cas13 protein described herein can be fused to a detectable moiety such as a fluorescent protein (e.g., green fluorescent protein or yellow fluorescent protein).

In accordance with the present invention, the Cas13 protein may be introduced as a protein as defined above, but alternatively the Cas13 protein may also be introduced in form of a nucleotide sequence encoding said protein. In other words, it may also be introduced in form of a nucleic acid molecule comprising a nucleotide sequence encoding said protein. It will be appreciated that the nucleotide sequence encodes said Cas13 protein in expressible form such that expression results in a functional Cas13 protein. Means and methods to ensure expression of a functional polypeptide are well known in the art. For said at least one Cas13 protein also at least one nucleotide sequence encoding said at least one Cas13 protein may be used. Meaning when e.g. two Cas13 proteins are comprised in said system, said one Cas13 protein is encoded by one nucleotide sequence and the other Cas13 protein is separately encoded by another nucleotide sequence. For example, the coding sequences may be comprised in a vector such as for example a plasmid, cosmid, virus, bacteriophage or another vector used conventionally e.g. in genetic engineering. The coding sequences inserted in the vector can e.g. be synthesized by standard methods, or isolated from natural sources. The coding sequences may further be ligated to transcriptional regulatory elements and/or to other amino acid encoding sequences. Such regulatory sequences are well known to those skilled in the art and include, without being limiting, regulatory sequences ensuring the initiation of transcription, internal ribosomal entry sites (IRES) (Owens, Proc. Natl. Acad. Sci. USA 98 (2001), 1471-1476) and optionally regulatory elements ensuring termination of transcription and stabilization of the transcript. Non-limiting examples for regulatory elements ensuring the initiation of transcription comprise a translation initiation codon, transcriptional enhancers such as e.g. the SV40-enhancer, insulators and/or promoters, such as for example the cytomegalovirus (CMV) promoter, SV40-promoter, RSV-promoter (Rous sarcome virus), the lacZ promoter, chicken beta-actin promoter, CAG-promoter (a combination of chicken betaactin promoter and cytomegalovirus immediate-early enhancer), the gai10 promoter, human elongation factor lot-promoter, AOX1 promoter, GAL1 promoter CaM-kinase promoter, the lac, trp or tac promoter, the lacUV5 promoter, the autographa californica multiple nuclear polyhedrosis virus (AcMNPV) polyhedral promoter or a globin intron in mammalian and other animal cells. Non-limiting examples for regulatory elements ensuring transcription termination include the V40-poly-A site, the tk-poly-A site or the SV40, lacZ or AcMNPV polyhedral polyadenylation signals, which are to be included downstream of the nucleic acid sequence of the invention. Additional regulatory elements may include translational enhancers, Kozak sequences and intervening sequences flanked by donor and acceptor sites for RNA splicing. Moreover, elements such as origin of replication, drug resistance gene or regulators (as part of an inducible promoter) may also be included. Nucleotide sequences encoding said Cas13 protein include DNA, such as cDNA or genomic DNA, and RNA. Preferably, embodiments reciting “RNA” are directed to mRNA.

Thus, the present invention comprises a composition comprising said CRISPR system comprising at least one DNA sequence encoding said at least one Cas13 protein as defined herein and at least one DNA sequence encoding said at least one gRNA as defined herein, which may refer to the DNA-based system. The present invention however also comprises a composition comprising said CRISPR system comprising at least one RNA sequence (mRNA sequence) encoding said at least one Cas13 protein as defined herein and at least one gRNA as defined herein, which may refer to the RNA-based system.

The nucleotide sequence encoding said Cas13 protein, in particular Cas13d, is advantageously codon optimized CRISPR protein. An example of a codon optimized sequence, is in this instance a sequence optimized for expression in eukaryotes, e.g., humans (i.e. being optimized for expression in humans), or for another eukaryote, animal or mammal as herein discussed. Whilst this is preferred, it will be appreciated that other examples are possible and codon optimization for a host species other than human, or for codon optimization for specific organs is known. In some embodiments, a nucleotide sequence encoding said Cas13 protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In general, codon optimization refers to a process of modifying a nucleotide sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/and these tables can be adapted in a number of ways. See Nakamura, Y, et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding said Cas13 protein correspond to the most frequently used codon for a particular amino acid.

It will be readily appreciated by the skilled person that more than one nucleotide sequence may encode a Cas13 protein, preferably Cas13d protein in accordance with the present invention due to the degeneracy of the genetic code. Degeneracy results because a triplet code designates 20 amino acids and a stop codon. Because four bases exist which are utilized to encode genetic information, triplet codons are required to produce at least 21 different codes. The possible 43 possibilities for bases in triplets give 64 possible codons, meaning that some degeneracy must exist. As a result, some amino acids are encoded by more than one triplet, i.e. by up to six. The degeneracy mostly arises from alterations in the third position in a triplet. This means that nucleotide sequences having different sequences, but still encoding the same Cas13 protein, can be employed in accordance with the present invention. The nucleotide sequences used in accordance with the present invention may be of natural as well as of (semi) synthetic origin. Thus, the nucleotide sequences may, for example, be nucleotide sequences that have been synthesised according to conventional protocols of organic chemistry. The person skilled in the art is familiar with the preparation and the use of said probes (see, e.g., Sambrook and Russel “Molecular Cloning, A Laboratory Manual”, Cold Spring Harbor Laboratory, N.Y. (2001)). Also in accordance with the present invention, the nucleotide sequences used in accordance with the invention may be nucleic acid mimicking molecules known in the art such as synthetic or semi-synthetic derivatives of nucleotide sequences and mixed polymers. They may contain additional non-natural or derivatised nucleotide bases, as will be readily appreciated by those skilled in the art. Nucleic acid mimicking molecules or derivatives according to the invention include, without being limiting, phosphorothioate nucleic acid, phosphoramidate nucleic acid, morpholino nucleic acid, hexitol nucleic acid (HNA), peptide nucleic acid (PNA) and locked nucleic acid (LNA).

gRNA

When the CRISPR protein is a Cas13 protein as it is the case in the present invention, a tracrRNA is not required in the composition as described elsewhere herein. However, said composition also additionally comprises at least one guide RNA (gRNA) capable of hybridizing with one or more target RNA molecules as defined herein.

As used herein, the term “guide RNA”, “gRNA”, or “complementary crRNA” refers to a polynucleotide comprising any polynucleotide sequence having sufficient complementarity with one or more target RNA molecules to hybridize with the one or more target RNA molecules and to direct sequence-specific binding of a RNA-targeting complex comprising the gRNA and a CRISPR Cas13 protein to the one or more target RNA molecule. In general, a gRNA may be any polynucleotide sequence (i) being able to form a complex with a CRISPR protein such as Cas13 and (ii) comprising a sequence having sufficient complementarity with a target polynucleotide sequence to hybridize with the target sequence and direct sequence-specific binding of a CRISPR complex to the target sequence. As used herein the term “gRNA capable of hybridizing” refers to a subsection of the gRNA having sufficient complementarity to the one or more target RNA molecules to hybridize thereto and to mediate binding of a CRISPR complex to the target RNA. In general, a guide sequence, which is part of the gRNA, is any polynucleotide sequence having sufficient complementarity with a target polynucleotide sequence as defined elsewhere herein to hybridize with the target sequence and direct sequence-specific binding of a CRISPR complex to the target sequence.

As used herein the term “capable of forming a complex with the Cas13 protein” refers to the gRNA having a structure that allows specific binding by the Cas13 protein to the gRNA such that a complex is formed that is capable of binding to a target RNA molecule in a sequence specific manner and that can exert a function on said target RNA. Structural components of the gRNA may include direct repeats and a guide sequence (or spacer). The sequence specific binding to the target RNA is mediated by a part of the gRNA, the “guide sequence”, being complementary to the target RNA.

In a further step such at least one gRNA were optimized by the present inventors. Complementary sequences of 22 bases are usually used as standard. The at least one gRNA or the nucleotide sequence encoding said gRNA as used herein and comprised by the composition of the present invention preferably comprise a length of at least about 23, such as at least about 24, at least about 25, at least about 26, at least about 27, at least about 28, at least about 29, or at least about 30 nucleotides. Thus, the gRNAs of the present invention have been optimized by extending their length as an additional modification to the novel composition as disclosed herein.

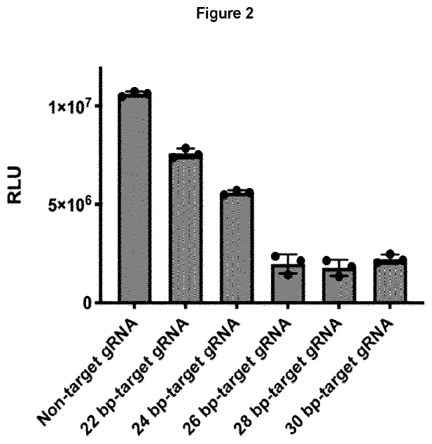

Said at least one gRNA or the nucleotide sequence encoding said gRNA comprised by the composition of the present invention comprises in a more preferred embodiment a length of between about 23 to about 30 nucleotides such as 23, 24, 25, 26, 27, 28, 29, or 30 nucleotides, preferably between about 24 to about 30 nucleotides, more preferably between about 25 to about 30 nucleotides, most preferably between about 26 to about 30 nucleotides. The inventors were able to show in particular that longer gRNAs comprising a length of between about 26 to about 30, between about 27 to about 30, between about 28 to about 30, between about 29 to about 30 nucleotides significantly increase the enzymatic activity of said Cas13 protein (see FIGS. 1 and 2). Preferably, the at least one gRNA has a length of about 26, 27, 28, 29, or about 30 nucleotides.

In a preferred embodiment, a composition comprising the CRISPR system comprising i) said at least one Cas13 protein or said nucleotide sequence encoding it being fused to said at least one NLS fused to said at least one NES and ii) at least one gRNA or said nucleotide sequence encoding it as defined herein, wherein said gRNA has a length of between about 26 to about 30 nucleotides is thus also envisaged herein. In another preferred embodiment, a composition comprising the CRISPR system comprising i) said at least one Cas13 protein or said nucleotide sequence encoding it being fused to said at least one NLS fused to said at least one NES and ii) at least one gRNA or said nucleotide sequence encoding it as defined herein, wherein said gRNA has a length of between about 27 to about 30 nucleotides is thus also envisaged herein. In another preferred embodiment, a composition comprising the CRISPR system comprising i) said at least one Cas13 protein or said nucleotide sequence encoding it being fused to said at least one NLS fused to said at least one NES and ii) at least one gRNA or said nucleotide sequence encoding it as defined herein, wherein said gRNA has a length of between about 28 to about 30 nucleotides is thus also envisaged herein. In another preferred embodiment, a composition comprising the CRISPR system comprising i) said at least one Cas13 protein or said nucleotide sequence encoding it being fused to said at least one NLS fused to said at least one NES and ii) at least one gRNA or said nucleotide sequence encoding it as defined herein, wherein said gRNA has a length of between about 29 to about 30 nucleotides is thus also envisaged herein. In an even more preferred embodiment, a composition comprising the CRISPR system comprising i) said at least one Cas13 protein or said nucleotide sequence encoding it being fused to said at least one NLS fused to said at least one NES and ii) at least one gRNA or said nucleotide sequence encoding it as defined herein, wherein said gRNA has a length of about 26 nucleotides is thus also envisaged herein. In another even more preferred embodiment, a composition comprising the CRISPR system comprising i) said at least one Cas13 protein or said nucleotide sequence encoding it being fused to said at least one NLS fused to said at least one NES and ii) at least one gRNA or said nucleotide sequence encoding it as defined herein, wherein said gRNA has a length of about 27 nucleotides is thus also envisaged herein. In another even more preferred embodiment, a composition comprising the CRISPR system comprising i) said at least one Cas13 protein or said nucleotide sequence encoding it being fused to said at least one NLS fused to said at least one NES and ii) at least one gRNA or said nucleotide sequence encoding it as defined herein, wherein said gRNA has a length of about 28 nucleotides is thus also envisaged herein. In another even more preferred embodiment, a composition comprising the CRISPR system comprising i) said at least one Cas13 protein or said nucleotide sequence encoding it being fused to said at least one NLS fused to said at least one NES and ii) at least one gRNA or said nucleotide sequence encoding it as defined herein, wherein said gRNA has a length of about 29 nucleotides is thus also envisaged herein. In another even more preferred embodiment, a composition comprising the CRISPR system comprising i) said at least one Cas13 protein or said nucleotide sequence encoding it being fused to said at least one NLS fused to said at least one NES and ii) at least one gRNA or said nucleotide sequence encoding it as defined herein, wherein said gRNA has a length of about 30 nucleotides is thus also envisaged herein.

In a preferred embodiment said at least one gRNA or the nucleotide sequence encoding said gRNA has at least about 80%, at least about 85%, at least about 90%, at least about 95%, at least about 96%, at least about 97%, at least about 98%, at least about 99% or even 100% complementary sequence identity to the one or more target RNA molecules. The inventors found out that having one mismatch with the one or more target RNA molecules could be tolerated, which would refer, depending on the length of the gRNA, to at least about 95% complementary sequence identity to the one or more target RNA molecules. Two adjacent mismatches result in a significant reduction in activity. If the two mismatches are not next to each other, the effect on activity is significantly less. However, if there are three mismatches with the one or more target RNA molecules, the at least one gRNA is not capable of hybridizing with the one or more target RNA molecules anymore. Preferably, said at least one gRNA or the nucleotide sequence encoding said gRNA has at least about 85% complementary sequence identity to the one or more target RNA molecules. In some embodiments, the degree of complementarity between a guide sequence as part of the gRNA as defined elsewhere herein and its corresponding target sequence comprised by the target RNA molecule, when optimally aligned using a suitable alignment algorithm, is about or more than about 80%, 85%, 90%, 95%, 96%, 97%, 98%, 99% or more, preferably about or more than about 85%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at novocraft.com), ELAND (Illumina, San Diego, Calif.), SOAP (available at soap.genomics.org.cn), and Maq (available at maq. sourceforge.net).

At least one (one or more), such as one, two, three, four, five and more gRNAs may be used in the composition according to the present invention. Thereby, each of the different gRNAs can bind to a viral or bacterial sequence in the genome. The simultaneous application of more than one different gRNAs that address several regions of the virus/bacterium genome may also be applied by the present invention. This refers to a process called “multiplexing”. Thus, even if the virus/bacterium mutates, efficient and sustained degradation can be ensured.

In certain embodiments, the at least one (one or more) gRNA is capable of binding/binds to the coding strand of the RNA. In certain embodiments, the gRNA is capable of binding/binds to the non-coding strand of the RNA. In certain embodiments, the gRNA binds to viral/bacterial genomic RNA (positive or negative sense or coding or non-coding strand). In certain embodiments, the gRNA binds to transcribed RNA (positive or negative sense or coding or non-coding strand) from viral/bacterial genomic DNA.

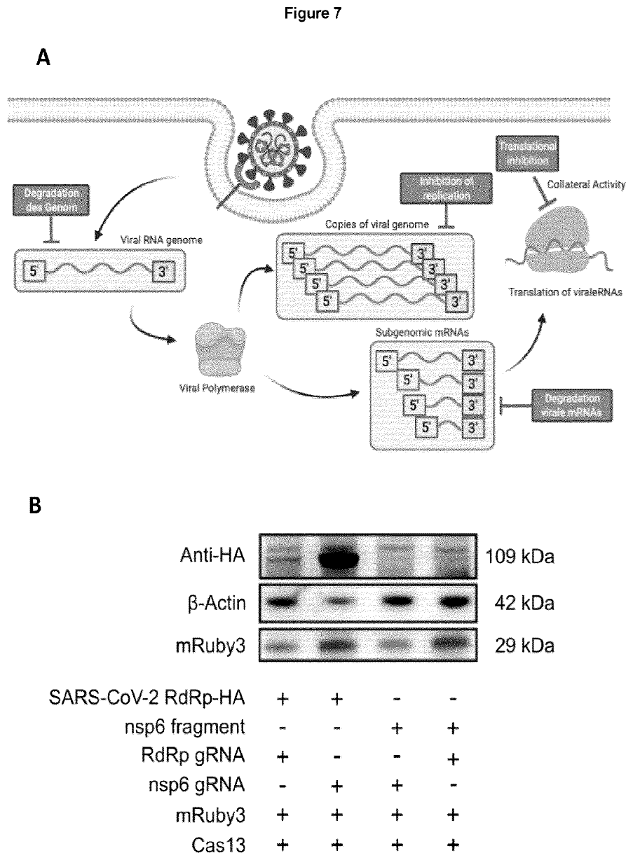

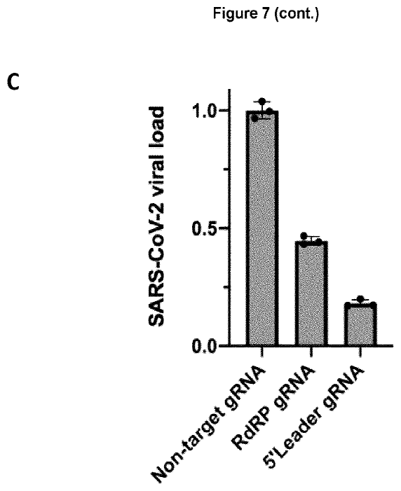

In a preferred embodiment, said at least one (one or more) gRNA is capable of hybridizing/binding or hybridizes/binds to a 5′- and/or 3′ untranslated region(s) of said one or more target RNA molecules (see FIG. 7a). The term “non-coding region”/“non-coding sequence” may be used interchangeably with the term “untranslated region”. Since these two areas are present both in the genome of any virus/bacterium and in all subgenomic mRNAs, the antiviral/antibacterial effect of Cas13 is strongest at these positions.

Moreover, said gRNA comprised by said composition may also be encoded by a nucleotide sequence. In other words, it may also be introduced in form of a nucleic acid molecule comprising a nucleotide sequence encoding said gRNA. The definitions and preferred embodiments recited above with regard to the nucleotide sequence encoding said Cas13 protein apply mutatis mutandis also to the nucleotide sequence encoding said gRNA.

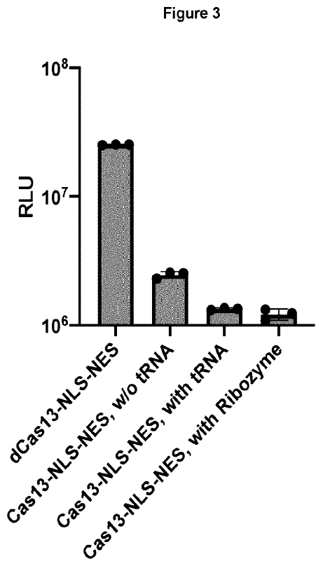

As an additional modification to said composition comprising said novel CRISPR system as described elsewhere herein, a fusion of the nucleotide sequence encoding said gRNA with one tRNA proves to be advantageous, since it supports the folding of the gRNA (see FIG. 3). Said tRNA is then fused directly as defined elsewhere herein to the 3′ end of the nucleotide sequence encoding said gRNA, preferably wherein said nucleotide sequence is DNA. Thus, a composition is also comprised herein, wherein the DNA nucleotide sequence encoding said gRNA is fused directly with a tRNA at the 3′ end of said nucleotide sequence. Any tRNA known to a person skilled in the art may be used. The predominance of selected tRNAs in a cell is generally a reflection of the codons used most frequently in peptide synthesis. Accordingly, genes can be tailored for optimal gene expression in a given organism based on codon optimization as defined elsewhere herein. Preferably the tRNA derived from murid γ-herpesvirus 68 (MHV68) having the amino acid sequence as depicted in SEQ ID NO: 8 is used for said fusion. In this context and also with regard to the fusion with a ribozyme, the definition of “derived” as defined elsewhere herein may be applied, also having at least about 50%, 60%, 70%, 75%, 80%, 85%, 90%, 95%, 96%, 97%, 98% or even at least about 99% RNA sequence identity with the tRNA or with the ribozyme from which it is derived. It is also comprised by the present invention that said tRNA may be fused to said gRNA as defined elsewhere herein via a linker as defined herein.

Alternatively, said nucleotide sequence encoding said gRNA, wherein said sequence preferably refers to a DNA sequence, can also be fused directly as defined herein to a ribonucleic acid enzyme (short: ribozyme). A “ribozyme” are RNA molecules that have the ability to catalyze specific biochemical reactions, including RNA splicing in gene expression as known to a person skilled in the art. A natural or even a synthetic ribozyme may be used herein for said fusion. In a preferred embodiment, said ribozyme is derived as defined elsewhere herein from Hepatitis delta virus (HDV) having the amino acid sequence as depicted in SEQ ID NO: 9. Contrary to the tRNA being used and defined herein, which may be split off by RNAse P and RNAse Z after a certain period of time, which results in a clean defined terminal end of said gRNA, preferably in a clean defined 3′-terminal end of said gRNA, the ribozyme being used herein excises itself from said gRNA, however also resulting in a clean defined terminal end of said gRNA, preferably in a clean defined 3′-terminal end of said gRNA—both modifications then leading to an efficiency improvement as described herein.

Also the subject of this invention are isolated ribozymes or ribozyme portions which are fused to said nucleotide sequence encoding said gRNA (see FIG. 3). Such ribozymes comprise a motif which specifically covalently fuses with said sequence, with the result that the ribozyme is joined specifically to the sequence. Ribozymes of the present invention can be a contiguous sequence or can be comprised of two noncontiguous components which interact with one another to form the complete ribozyme. The two noncontiguous components are a ribozyme segment which specifically covalently fuses to said sequence and a ribozyme segment which comprises the remaining ribozyme.

The ribozyme RNA which specifically covalently fuses to said nucleotide sequence is present in (is a component or segment of) a ribozyme which is either a contiguous sequence (the entire ribozyme is a contiguous sequence) or comprised of two noncontiguous components: one which comprises the ribozyme RNA which specifically covalently fuses to said sequence and one which comprises the remainder of the ribozyme sequence. The remaining ribozyme sequence is the ribozyme sequence which, in combination with the component which covalently fuses to said sequence, makes up the complete ribozyme. The ribozyme sequence or segment which covalently fuses to said sequence can be as short as one nucleotide in length and can be from any location (e.g., 5′ end, internal segment, 3′ end) in the ribozyme. In one embodiment, the ribozyme segment includes from one to about 18 nucleotides, such as from the first to about the 18th nucleotide (from the 5′ end) of a ribozyme. In further embodiments, the ribozyme segment is the first 13 to 18 nucleotides (from the 5′ end) of the ribozyme. (e.g., the first 13, 14, 15, 16, 17, or 18 nucleotides from the 5′ end). The other component is the remaining ribozyme sequence (the remainder of the ribozyme which is necessary to form a functional ribozyme.). The two ribozyme components interact with one another to form a functional (complete) ribozyme. The 5′ end of the ribozyme RNA optionally comprises three phosphate groups or an mRNA cap, such as a 7-methyl guanosine triphosphate. Thus, it is also comprised herein, that said nucleotide sequence encoding said gRNA is covalently fused with a ribozyme RNA as defined herein. Preferably, said ribozyme as defined elsewhere herein is fused directly as defined elsewhere herein to the 3′ end of the nucleotide sequence encoding said gRNA, preferably wherein said nucleotide sequence is DNA. Thus, a composition is also comprised herein, wherein the DNA nucleotide sequence encoding said gRNA is fused directly with a ribozyme as defined elsewhere herein at the 3′ end of said nucleotide sequence.

As mentioned above, the fusion of said tRNA and/or said ribozym, which is a direct fusion to the DNA nucleotide sequence encoding said gRNA, may be covalently. The present invention thus further comprises that said nucleotide sequence (DNA sequence) encoding said gRNA is (directly) fused covalently with a tRNA and/or a ribozyme. In this context, when the term “covalently fused” is used, a covalent bond is formed between the nucleotide sequence encoding said gRNA of the present invention and the tRNA or the ribozyme as defined above. In these embodiments, the term “directly” again means that the nucleotide sequence encoding said gRNA and the tRNA and/or ribozyme are arranged one after the other without a linker as defined elsewhere herein.

In a preferred embodiment, a composition comprising the CRISPR system comprising i) a nucleotide sequence encoding said at least one Cas13 protein being fused to said at least one NLS fused to said at least one NES and ii) a nucleotide sequence encoding said at least one gRNA as defined herein, wherein said gRNA has a length of between about 26 to about 30 nucleotides and wherein said nucleotide sequence encoding said gRNA is also fused to a tRNA or a ribozyme as defined is thus also envisaged herein. In another preferred embodiment, a composition comprising the CRISPR system comprising i) a nucleotide sequence encoding said at least one Cas13 protein being fused to said at least one NLS fused to said at least one NES and ii) a nucleotide sequence encoding said at least one gRNA as defined herein, wherein said gRNA has a length of between about 27 to about 30 nucleotides and wherein said nucleotide sequence encoding said gRNA is also fused to a tRNA or a ribozyme as defined is thus also envisaged herein. In another preferred embodiment, a composition comprising the CRISPR system comprising i) a nucleotide sequence encoding said at least one Cas13 protein being fused to said at least one NLS fused to said at least one NES and ii) a nucleotide sequence encoding said at least one gRNA as defined herein, wherein said gRNA has a length of between about 28 to about 30 nucleotides and wherein said nucleotide sequence encoding said gRNA is also fused to a tRNA or a ribozyme as defined is thus also envisaged herein. In another preferred embodiment, a composition comprising the CRISPR system comprising i) a nucleotide sequence encoding said at least one Cas13 protein being fused to said at least one NLS fused to said at least one NES and ii) a nucleotide sequence encoding said at least one gRNA as defined herein, wherein said gRNA has a length of between about 29 to about 30 nucleotides and wherein said nucleotide sequence encoding said gRNA is also fused to a tRNA or a ribozyme as defined is thus also envisaged herein. In an even more preferred embodiment, a composition comprising the CRISPR system comprising i) a nucleotide sequence encoding said at least one Cas13 protein being fused to said at least one NLS fused to said at least one NES and ii) a nucleotide sequence encoding said at least one gRNA as defined herein, wherein said gRNA has a length of about 26 nucleotides and wherein said nucleotide sequence encoding said gRNA is also fused to a tRNA or a ribozyme as defined is thus also envisaged herein. In another even more preferred embodiment, a composition comprising the CRISPR system comprising i) a nucleotide sequence encoding said at least one Cas13 protein being fused to said at least one NLS fused to said at least one NES and ii) a nucleotide sequence encoding said at least one gRNA as defined herein, wherein said gRNA has a length of about 27 nucleotides and wherein said nucleotide sequence encoding said gRNA is also fused to a tRNA or a ribozyme as defined is thus also envisaged herein. In another even more preferred embodiment, a composition comprising the CRISPR system comprising i) a nucleotide sequence encoding said at least one Cas13 protein being fused to said at least one NLS fused to said at least one NES and ii) a nucleotide sequence encoding said at least one gRNA as defined herein, wherein said gRNA has a length of about 28 nucleotides and wherein said nucleotide sequence encoding said gRNA is also fused to a tRNA or a ribozyme as defined is thus also envisaged herein. In another even more preferred embodiment, a composition comprising the CRISPR system comprising i) a nucleotide sequence encoding said at least one Cas13 protein being fused to said at least one NLS fused to said at least one NES and ii) a nucleotide sequence encoding said at least one gRNA as defined herein, wherein said gRNA has a length of about 29 nucleotides and wherein said nucleotide sequence encoding said gRNA is also fused to a tRNA or a ribozyme as defined is thus also envisaged herein. In another even more preferred embodiment, a composition comprising the CRISPR system comprising i) a nucleotide sequence encoding said at least one Cas13 protein being fused to said at least one NLS fused to said at least one NES and ii) a nucleotide sequence encoding said at least one gRNA as defined herein, wherein said gRNA has a length of about 30 nucleotides and wherein said nucleotide sequence encoding said gRNA is also fused to a tRNA or a ribozyme as defined is thus also envisaged herein.

The guide RNA(s) nucleotide sequences and/or Cas nucleotide sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). Suitable promoter have already been mentioned previously and can be further selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the (3-actin promoter, the phosphoglycerol kinase (PGK) promoter, the EF1α promoter and U6 promoter, TetOn/Off, 7SK, CAG, CBh, UbC or any tetracycline-dependent or interferon-dependent promoter.

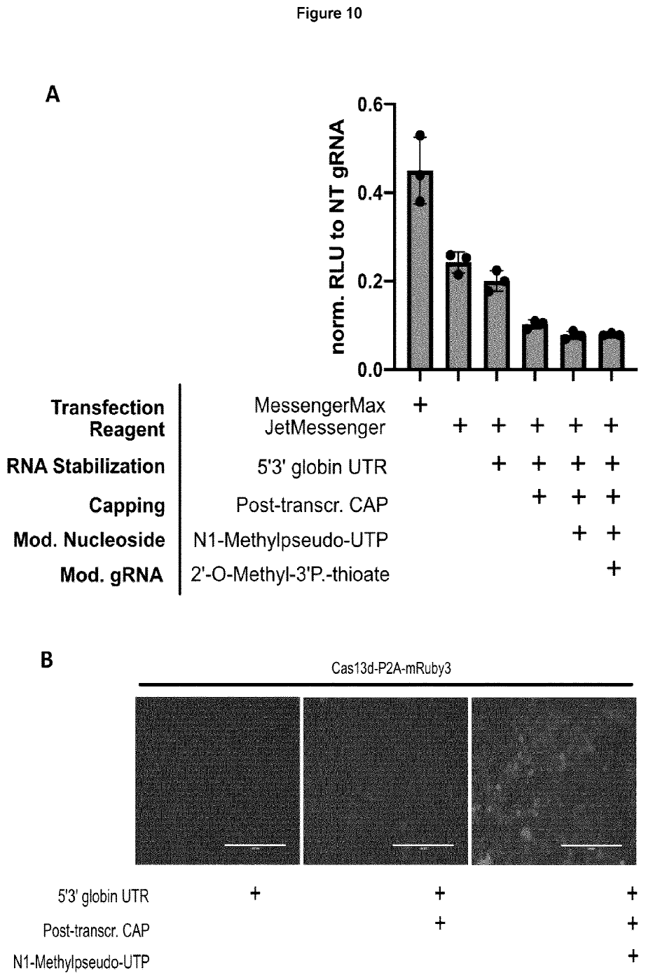

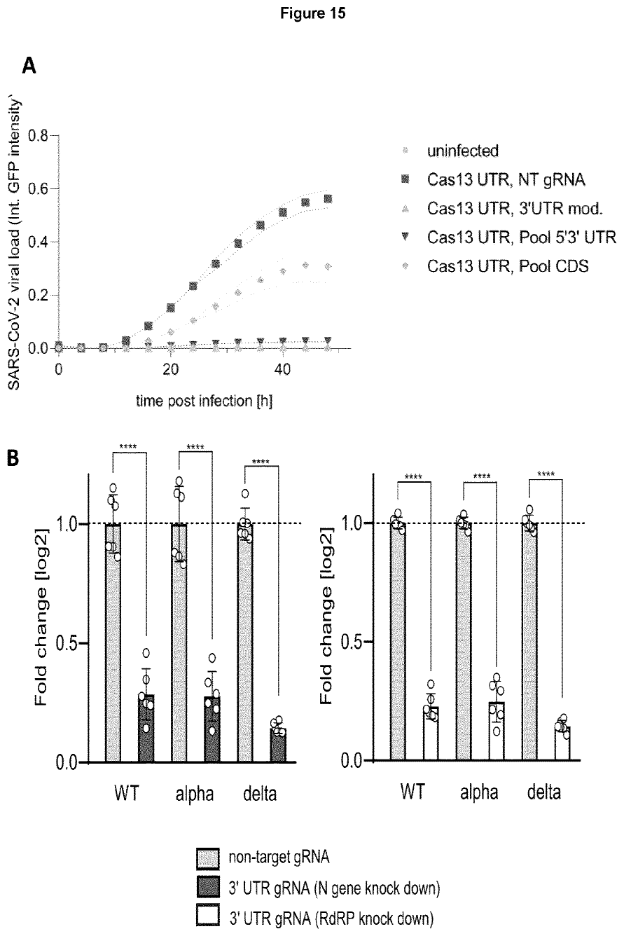

Also comprised by the present invention is the composition comprising said CRISPR system as defined elsewhere herein, wherein the nucleotide sequence encoding said Cas13 protein and/or said gRNA as defined elsewhere herein may further be modified on the RNA level. In other words, the RNA sequences encoding said Cas13 protein and/or said gRNA as defined herein may comprise modified UTRs (see FIG. 15), the post- or cotranscriptional addition of a 5′ CAP structure, preferably a posttranscriptional addition of a 5′CAP structure, the replacement of UTP by N1-methylpseudo-UTP, and/or the replacement of the two 5′ and/or 3′ terminal nucleotides of said gRNA by 2′-O-methyl-3′P-thioate (see FIG. 10A). Also comprised herein and to enhance expression and reduce possible toxicity, the nucleotide sequences encoding said Cas13 protein and/or said guide RNA as defined elsewhere herein can alternatively or additionally to the above mentioned modifications be modified to include one or more modified nucleoside e.g., using pseudo-U and/or 5-methyl-C. In a preferred embodiment the present invention comprises the composition comprising said CRISPR system as defined elsewhere herein, wherein the nucleotide sequence encoding said gRNA as defined elsewhere herein is further modified by the replacement of the two 5′ and/or 3′ terminal nucleotides of said gRNA by 2′-O-methyl-3′P-thioate (also called 2′-O-methyl phosphorothioate).

Using modified UTRs 5′ and/or 3′ UTRs from beta-globin polyA tail (a string of 120 or more adenines) may stabilize said RNAs. Additionally, said post- or cotranscriptional five-prime cap (5′ CAP) being a specially altered nucleotide on the 5′ end of RNA transcripts of the present invention protects the mRNA from degradation by exonucleases and may also be important for ejection from the nucleus and initiation of translation. Another key advancement can be seen in the preparation of chemically modified mRNAs, by replacing standard bases by modified ones, e.g. uridine with pseudouridine. Thereby, the half-life of the mRNA, the translation efficiency and its immunological profile is improved. N1-methyl-pseudouridine results in more efficient translation and at the same time represses innate immune responses in comparison to the unmodified mRNA and is thus preferred. The last modification by which the gRNA may be stabilized (the replacement of the two 5′ and/or 3′ terminal nucleotides of said gRNA by 2′-O-methyl-3′P-thioate) includes that the 2′OH on the sugar of the RNA may be replaced by a methyl group and that the phosphodiester bond in the RNA backbone may be modified by sulfur, whereby the gRNA becomes resistant to RNAses. Any combination of each of the above mentioned modifications to said RNAs may also be comprised herewith.

These modifications of said RNAs (Cas13 mRNA and/or gRNA) improved knockdown-efficiency for RNA delivery of Cas13 protein (see FIG. 10B or FIG. 15). Thus, by applying the additional modifications to said RNAs encoding said Cas13 protein and/or said gRNA as defined herein, which are comprised by said CRISPR system being comprised in the composition of the present invention, may again lead to an increased knockdown efficiency of the Cas13 protein as it has been described by the present invention for the other modifications being used as additional modifications applied to said system besides the fusion of at least one NLS fused to at least one NES. Thus, said RNA sequences of the CRISPR system comprised by the composition of the present invention, which encode said Cas13 protein and/or said gRNA as defined herein may additionally (in addition to said other modifications of said system as mentioned by the present invention) comprise any modification selected from the group consisting of modified UTRs as defined herein, modified nucleosides e.g., using pseudo-U and/or 5-methyl-C, a post- or cotranscriptional (preferably posttranscriptional) 5′ CAP structure, the replacement of UTP by N1-Methylpseudo-UTP and the replacement of the two 5′ and/or 3′ terminal nucleotides of said gRNA by 2′-O-methyl-3′P-thioate. In a particular embodiment, said RNA sequences of the CRISPR system comprised by the composition of the present invention, which encode said Cas13 protein and/or said gRNA as defined herein may additionally (in addition to said other modifications of said system as mentioned by the present invention) comprise modified 3′ UTRs, specifically comprising 3′ UTR having N gene of SARS-CoV-2 knock down and/or RdRP gene knock down (see FIG. 15).

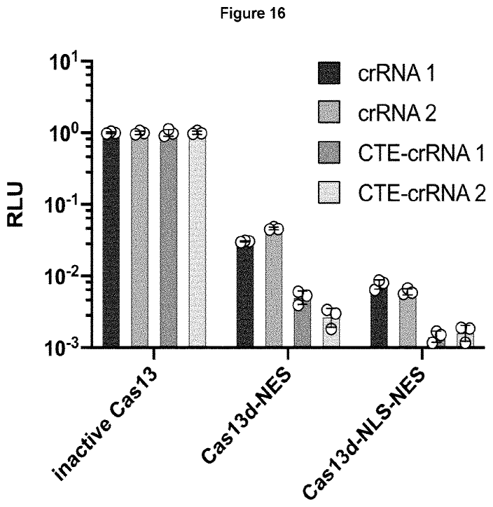

Also comprised by the present invention is the composition comprising said CRISPR system as defined elsewhere herein, wherein the nucleotide sequence encoding said Cas13 protein and/or said gRNA as defined elsewhere herein may further be modified on the DNA level, which includes fusing the nucleotide sequence encoding gRNA, preferably wherein the nucleotide sequence is DNA, with at least one viral export element. A fusion of the nucleotide sequence encoding said gRNA with at least one viral export element as defined herein proves to be advantageous, since it increases the knockdown efficiency even more (see FIG. 16). Said at least one viral export element as defined herein is then fused as defined elsewhere herein to the 5′ end of the nucleotide sequence encoding said gRNA, preferably wherein said nucleotide sequence is DNA. In this context, fusing means that the nucleotide sequence encoding said gRNA and the at least one viral export element are fused via a linker, such as any nucleotide linker. Thus, a composition is also comprised herein, wherein the DNA nucleotide sequence encoding said gRNA is fused via a linker such as any nucleotide linker with at least one viral export element as defined herein at the 5′ end of said nucleotide sequence. The term “at least one viral export element” means that also two, three, four, five or more viral export elements as defined herein may be used. In a preferred embodiment, said at least one viral export element is a CTE or a VARdm, preferably a CTE, which is fused as defined herein with the nucleotide sequence encoding said gRNA, which is preferably a DNA sequence. In sum, the combination of NLS-NES and CTE-gRNA maximizes the knockdown efficiency. Such fusion to the gRNA can also be performed on the protein level of said composition.

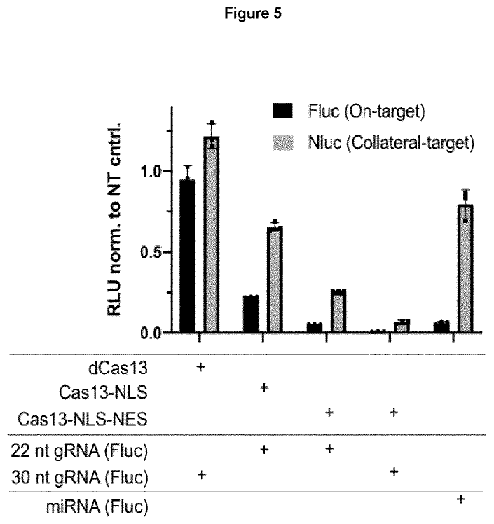

Additionally, the present inventors also found out that said composition comprising said novel CRISPR system as defined elsewhere herein has collateral RNAse-activity in mammalian cells (see FIG. 5). This led to the conclusion that said modified system of the composition comprising said at least one Cas13 protein may additionally have an unspecific RNAse-activity, which goes beyond the effect against the complementary target sequences which are hybridized by said at least one RNAs/cleaved by said Cas13 protein and which is directed against ribosomal RNAs (rRNAs), among others. In other words, not only the bound target RNA is cleaved/degraded, but also bystander effects occur (so-called collateral activity), in which neighboring RNAs (mainly rRNAs) are also degraded. Said additional activity of said system comprised by said composition may be present in all kinds of mammalian cells. Examples for such mammalian cells can be mouse neuroblastoma (N2a), mouse myoblast (C2C12), human glioblastoma (U87M), human liver cancer cells (HepG2), mouse micoglia cells (BV-2), or primary cells.

The cleavage of viral or bacterial rRNA as the collateral target besides the one or more target RNA molecules as defined herein by said system according to the present invention is even enhanced by applying the novel system as modified herein. The formation of essential structural proteins of the virus or bacterium is thus additionally reduced by such a collateral activity of said system comprised by the composition. This is regarded as an additional mechanism, in order to lower the e.g. virus load by slowing down the virus replication in the cell further (see FIG. 5).

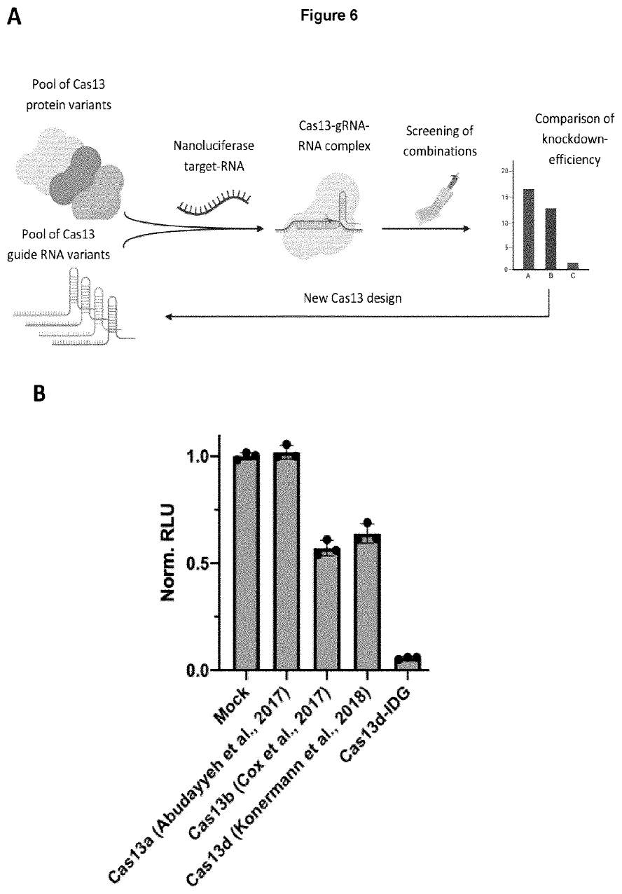

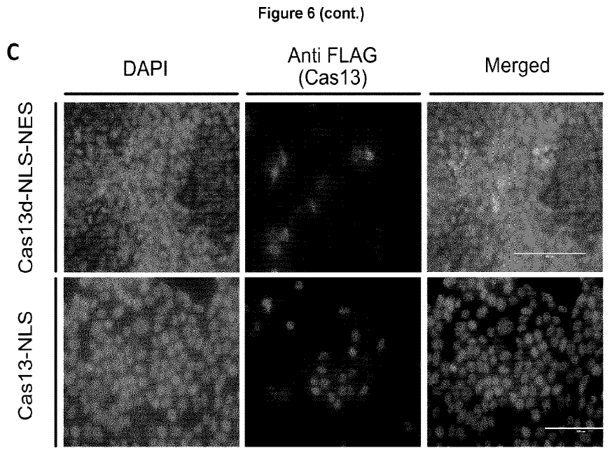

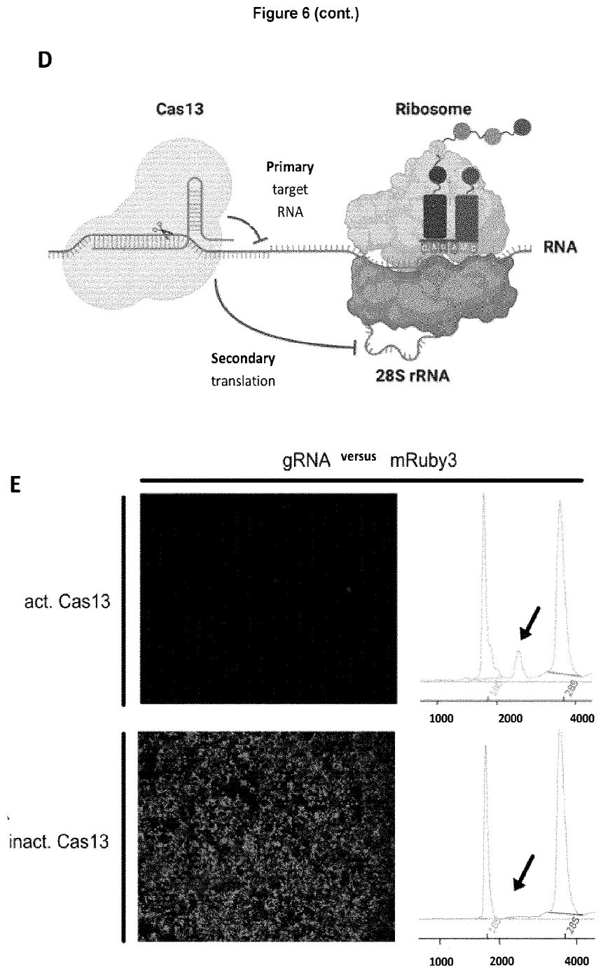

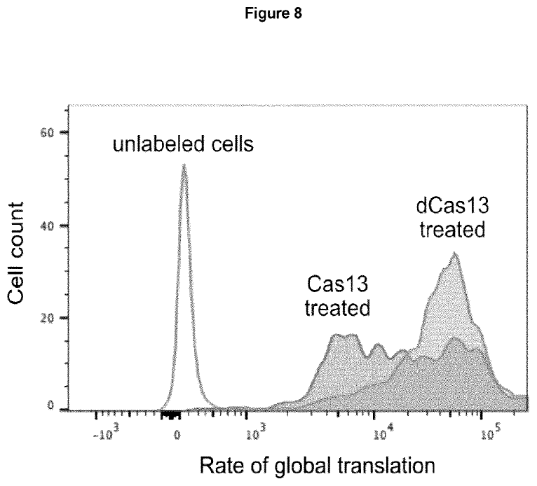

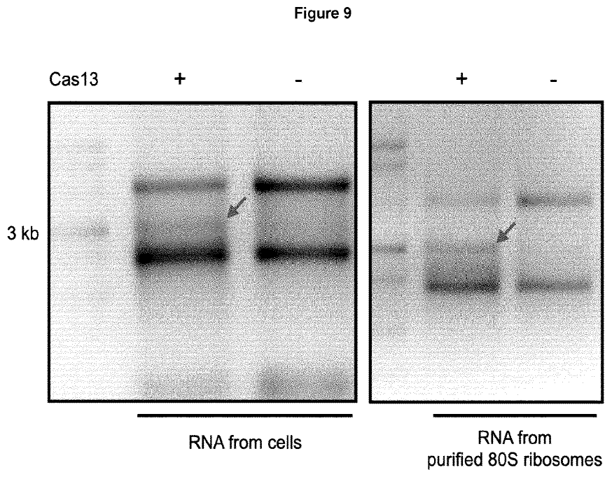

Such collateral RNAse activity of the Cas13 protein against mainly rRNA is shown by the specific binding to 28S rRNA. In other words, the composition comprising said particular system as defined herein is capable of binding and cleaving 28S rRNA. The reduction of ribosomal RNAs, which has been experimentally proven by the inventors, may lead to a temporary translation blockage in the cell due to a reduced amount of ribosomes, which means that said virus or bacterium can also be attacked on a further level as well. (see FIG. 8). Thus, said system comprised in the composition may have a preference for a defined position of the 28S ribosomal RNA, which is why said Cas13 protein may first cut its target RNA. Only when a target RNA is found the Cas13 protein is activated and also may cut a defined position of the ribosomal RNA, which ultimately leads to a transient inhibition of protein translation (see also FIG. 6D and E; FIG. 9).

The CRISPR protein such as Cas13 protein and/or any of the present RNAs, for instance a guide RNA, can be delivered using any suitable vector, e.g., plasmid or viral vectors, such as adeno associated virus (AAV), lentivirus, adenovirus or other viral vector types, or combinations thereof. Such proteins and one or more guide RNAs can be packaged into one or more vectors, e.g., plasmid or viral vectors. In some embodiments, the vector, e.g., plasmid or viral vector is delivered to the tissue of interest by, for example, an intramuscular injection, while other times the delivery is via intravenous, transdermal, intranasal, oral, mucosal, or other delivery methods. Such delivery may be either via a single dose, or multiple doses. One skilled in the art understands that the actual dosage to be delivered herein may vary greatly depending upon a variety of factors, such as the vector choice, the target cell, organism, or tissue, the general condition of the subject to be treated as mentioned elsewhere herein, the degree of transformation/modification sought, the administration route, the administration mode, the type of transformation/modification sought, etc. Such a dosage may further contain a carrier as defined elsewhere herein.

In one embodiment herein the delivery of said composition comprising said system based on a DNA level is via an adenovirus, which may be at a single booster dose. In another embodiment herein, the adenovirus is delivered via multiple doses. In a further embodiment herein the delivery of said composition comprising said system based on a DNA level is via a plasmid. In such plasmid compositions, the dosage should be a sufficient amount of plasmid to elicit a response. For instance, suitable quantities of plasmid DNA in plasmid compositions can be from about 0.1 to about 2 mg, or from about 1 μg to about 10 μg per 70 kg individual. Plasmids of the invention will generally comprise (i) a promoter; (ii) a sequence encoding a Cas13 protein as defined herein, operably linked to said promoter; (iii) a selectable marker; (iv) an origin of replication; and (v) a transcription terminator downstream of and operably linked to (ii). The plasmid preferably also encodes the RNA components of a CRISPR complex such as comprising a nucleotide sequence encoding said gRNA as defined herein, but one or more of these may instead be encoded on a different vector. In another further embodiment herein the delivery of said composition comprising said system based on a DNA level is via a particle and/or a nanoparticle as defined elsewhere herein.

In some embodiments said composition comprising said system based on a RNA level of the invention (including e.g. mRNA encoding said Cas13 protein and already chemically synthesized gRNA) and also on a protein level (including Cas13 protein and already chemically synthesized gRNA) are delivered in liposome or lipofectin formulations and the like and can be prepared by methods well known to those skilled in the art. Such methods are described, for example, in U.S. Pat. Nos. 5,593,972, 5,589,466, and 5,580,859, which are herein incorporated by reference. Thus, delivery of the nucleotide sequence encoding Cas13 protein and/or delivery of the gRNAs of the invention may be in RNA form and via liposomes, particles microvesicles. For example, Cas13 mRNA and gRNA can be packaged into liposomal particles for delivery in vivo.

Liposomes are spherical vesicle structures composed of a uni- or multilamellar lipid bilayer surrounding internal aqueous compartments and a relatively impermeable outer lipophilic phospholipid bilayer. Liposomes have gained considerable attention as drug delivery carriers because they are biocompatible, nontoxic, can deliver both hydrophilic and lipophilic drug molecules, protect their cargo from degradation by plasma enzymes, and transport their load across biological membranes and the blood brain barrier (BBB). Liposomes can be made from several different types of lipids; however, phospholipids are most commonly used to generate liposomes as drug carriers. Liposomal transfection reagents such as lipofectamine from Life Technologies and other reagents on the market can effectively deliver RNA molecules into the liver.

In general, a particle is defined as a small object that behaves as a whole unit with respect to its transport and properties. Particles are further classified according to diameter. Coarse particles cover a range between 2,500 and 10,000 nanometers. Fine particles are sized between 100 and 2,500 nanometers. Ultrafine particles, or nanoparticles, are generally between 1 and 100 nanometers in size. The basis of the 100-nm limit is the fact that novel properties that differentiate particles from the bulk material typically develop at a critical length scale of under 100 nm. Particles delivery systems within the scope of the present invention may be provided in any form, including but not limited to solid, semi-solid, emulsion, or colloidal particles. As such any of the delivery systems described herein, including but not limited to, e.g., lipid-based systems, liposomes, micelles, microvesicles, exosomes, or gene gun may be provided as particle delivery systems within the scope of the present invention. In general, a “nanoparticle” refers to any particle having a diameter of less than 1000 nm.

Means of delivery of RNA/protein also preferred include delivery of RNA/protein via nanoparticles (Cho, S., Goldberg, et al. Advanced Functional Materials, 19: 3112-3118, 2010) or exosomes (Schroeder, A., et al., Journal of Internal Medicine, 267: 9-21, 2010). In certain preferred embodiments, nanoparticles of the invention have a greatest dimension (e.g., diameter) of 500 nm or less. In other preferred embodiments, nanoparticles of the invention have a greatest dimension ranging between 25 nm and 200 nm. In other preferred embodiments, particles of the invention have a greatest dimension of 100 nm or less. In other preferred embodiments, nanoparticles of the invention have a greatest dimension ranging between 35 nm and 60 nm. Exosomes are endogenous nano-vesicles that transport RNAs and proteins, and which can deliver RNA to the brain and other target organs. Indeed, exosomes have been shown to be particularly useful in delivery siRNA, a system with some parallels to the RNA-targeting system. More details on the delivery systems with regard to DNA delivery (AAV, lentivirus etc.) or RNA/protein delivery (liposome, particles, exosomes etc.) are described in US20200165594, which is herein incorporated by reference.

CRISPR Cas13 mRNA and gRNA might also be delivered separately. CRISPR Cas13 mRNA can be delivered prior to the gRNA to give time for CRISPR Cas13 protein to be expressed. CRISPR Cas13 mRNA might be administered 1-12 hours (preferably around 2-6 hours) prior to the administration of gRNA. Alternatively, CRISPR Cas13 mRNA and gRNA can be administered together. Advantageously, a second booster dose of gRNA can be administered 1-12 hours (preferably around 2-6 hours) after the initial administration of CRISPR Cas13 mRNA+gRNA.

CRISPR Cas13 protein and gRNA might also be delivered separately. Preferably, CRISPR Cas13 protein and gRNA are delivered together. Before said delivery said protein and said gRNA may be mixed and freezed as a complex until administration of said complex. Advantageously, a second booster dose of gRNA can be administered 1-12 hours (preferably around 2-6 hours) after the initial administration of CRISPR Cas13 protein+gRNA.

### Target RNA Molecules

As already mentioned herein, said at least one gRNA is capable of hybridizing with one or more target RNA molecules. In the context of formation of a CRISPR complex, “target sequence” refers to a sequence to which a guide sequence (part of the gRNA) is designed to have complementarity as defined elsewhere herein, where hybridization between a target sequence and a guide sequence promotes the formation of a CRISPR complex. A target sequence may comprise RNA polynucleotides. The term “target RNA molecules” refers to a RNA polynucleotide (target RNA) being or comprising the target sequence. In other words, the target RNA may be a RNA polynucleotide or a part of a RNA polynucleotide to which a part of the gRNA, i.e. the guide sequence, is designed to have complementarity as described herein and to which the effector function mediated by the complex comprising CRISPR effector protein and a gRNA is to be directed. In some embodiments, a target RNA molecule and thus a target RNA sequence is located in the nucleus and/or cytoplasm of a cell. The target RNA, i.e. the RNA of interest, is the RNA to be targeted by the present invention leading to the recruitment to, and the binding of the Cas13 protein at, the target site of interest on the target RNA. The one or more target RNA molecules may be any suitable form of RNA. This may include, in some embodiments, mRNA. In other embodiments, the one or more target RNA molecules may include tRNA or rRNA. In other embodiments, the one or more target RNA molecules may include miRNA. In other embodiments, the one or more target RNA molecules may include siRNA.

In some embodiments, the one or more target RNA molecules comprising the target sequence or also the target sequence per se may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non coding RNA (ncRNA), long non-coding RNA (lncRNA), and small cytoplasmatic RNA (scRNA). In some preferred embodiments, one or more target RNA molecules comprising the target sequence or also the target sequence per se may be a sequence within a RNA molecule selected from the group consisting of mRNA, pre-mRNA, and rRNA. In some other preferred embodiments, the one or more target RNA molecules comprising the target sequence or also the target sequence per se may be a sequence within a RNA molecule selected from the group consisting of ncRNA, and lncRNA. In some other preferred embodiments, the one or more target RNA molecules comprising the target sequence or also the target sequence per se may be a sequence within an mRNA molecule or a pre-mRNA molecule.

Thus, the at least one gRNA capable of hybridizing as defined elsewhere herein with one or more target RNA molecules may also comprise hybridization with one or more RNA target sequence. Preferably, by targeting the one or more target RNA molecules/RNA target sequences the at least one gRNA that are complementary to a certain extent as defined elsewhere herein to said molecules/target sequences focus on the 5′ and/or 3′ untranslated regions/parts of said RNA molecules (mRNA molecules) as already defined elsewhere herein.

In a preferred embodiment, said one or more target RNA molecules are viral or bacterial target RNA molecules. In particular embodiments, the virus is an RNA virus. In further embodiments, the virus is a single stranded or double stranded RNA virus. In further embodiments, the virus is a positive sense RNA virus or a negative sense RNA virus or an ambisense RNA virus. In further embodiments, the virus is a Retroviridae virus, Lentiviridae virus, Coronaviridae virus, a Picornaviridae virus, a Caliciviridae virus, a Flaviviridae virus, a Togaviridae virus, a Bomaviridae, a Filoviridae, a Paramyxoviridae, a Pneumoviridae, a Rhabdoviridae, an Arenaviridae, a Bunyaviridae, an Orthomyxoviridae, or a Deltavirus. In particular embodiments, the virus is selected from the group consisting of Lymphocytic choriomeningitis virus, Coronavirus, HIV, SARS, Venezuelan Equine Encephalitis (VEE) virus, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Boma disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza and Hepatitis D virus. In another particular embodiments, the virus is a DNA virus. In further embodiments, the virus is a single stranded or double stranded DNA virus. In further embodiments, the virus is a positive sense DNA virus or a negative sense DNA virus or an ambisense DNA virus. In further embodiments, the virus is a Myoviridae, Podoviridae, Siphoviridae, Alloherpesviridae, Herpesviridae (including human herpes virus, and Varicella Zorter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, or Rhizidovirus. In certain example embodiments, the virus may be a retrovirus. Example retroviruses may include one or more of or any combination of viruses of the Genus Alpharetrovirus, Betaretrovirus, Gammaretrovirus, Deltaretrovirus, Epsilonretrovirus, Lentivirus, Spumavirus, or the Family Metaviridae, Pseudoviridae, and Retroviridae (including HIV), Hepadnaviridae (including Hepatitis B virus), and Caulimoviridae (including Cauliflower mosaic virus). In certain embodiments, the virus is a drug resistant virus and thus belongs to a multi-resistant germ as defined elsewhere herein. By means of example, and without limitation, the virus may be a ribavirin resistant virus. Ribavirin is a very effective antiviral that hits a number of RNA viruses. In a more preferred embodiment the term “viral” as used throughout the present invention refers to a coronavirus, influenza A virus, ebola virus, morbilivirus, hepacivirus, flavivirus such as TBE virus, dengue virus, yellow fever virus and/or zika virus.

With regard to bacterial target RNA molecules, any RNA molecules as defined herein from any bacterium may be comprised which may be hybridized by said gRNA as defined herein. Since bacteria also comprise RNA, it is plausibly demonstrated that said composition comprising said CRISPR system can also be applied to target one or more bacterial RNA molecules. In certain embodiments, the bacterium is a drug resistant bacterium and thus belongs to a multi-resistant germ. By means of example, and without limitation, the resistant bacterium may be Staphylococcus aureus, Streptococcus pneumoniae, Koagulase-negative Staphylokokken, Enterococcus faecium, Enterococcus faecalis, Escherichia coli, Klebsiella pneumoniae, Enterobacter cloacae, Serratia marcescens, Citrobacter spp., Pseudomonas aeruginosa, Acinetobacter baumannii and Stenotrophomonas maltophilia. Such multi-resistant germ as used herein refers to a species of microorganism (such as a virus or bacterium), which comprise antimicrobial resistance to at least one antimicrobial drug as known to a person skilled.

Thus, also comprised by the present invention is that said composition as defined herein is suitable for inactivating bacterial or viral single-stranded (ss) RNA. In this context, the term “inactivating” or “inactivate” or “inactivation” refers to the composition comprising said CRISPR system as defined herein, wherein said the at least one Cas13 protein forms a complex with the at least one gRNA and wherein the at least one gRNA directs the complex to the one or more target RNA molecules, which not only targets/detects the one or more target RNA molecules, but then also cleaves/degrades said target RNA molecules. Thus, the term “cleaving”, “cleave”, “cleavage” or “degrading”, “degrade” or “degradation” can also be used interchangeably with the term “inactivating”, “inactivate” or “inactivation”.

### Pharmaceutical Composition

According to the present invention, the composition as defined throughout the present invention may further comprise at least one carrier as defined elsewhere herein. The present invention also relates to said composition as defined herein, which is a pharmaceutical composition. Said pharmaceutical composition is thus used herein for therapeutic purposes. Moreover, the present invention relates to the use of said composition as disclosed herein above for the preparation of a pharmaceutical composition.

In accordance with the present invention, the term “pharmaceutical composition” relates to a composition for administration to a patient, preferably a human patient. Pharmaceutical compositions or formulations are usually in such a form as to allow the biological activity of the active ingredient to be effective and may therefore be administered to a subject for therapeutic use as described herein. The pharmaceutical composition can be administered by inhalation, injection, infusion, or orally. Thus, the pharmaceutical composition may be a composition for oral, parenteral, trans-dermal, intra-luminal, intra-arterial, intra-venous, intra-thecal and/or intranasal administration or for direct injection into tissue. The pharmaceutical compositions can be administered to the subject at a suitable dose. The dosage regimen will be determined by the attending physician and by clinical factors. As is well known in the medical arts, dosages for any one patient depend upon many factors, including the patient's size, body surface area, age, the particular compound to be administered, sex, time and route of administration, general health, and other drugs being administered concurrently.

The present invention may also encompass the pharmaceutical composition as defined herein, further comprising at least one pharmaceutically acceptable carrier. Hence, the therapeutic composition of the present invention further comprises a pharmaceutically acceptable carrier, diluent or excipient. Said terms can be used interchangeably. Said pharmaceutically acceptable carrier (also called excipient or diluent) includes any excipient/carrier/diluent that does not itself elicit an adverse reaction harmful to the subject receiving the pharmaceutical composition.

Suitable carriers are typically large, slowly metabolized macromolecules such as proteins, polysaccharides, polylactic acids, polyglycolic acids, polymeric amino acids, amino acid copolymers and lipid aggregates such as, e.g. oil droplets or liposomes. The carrier used in combination with the (pharmaceutical) composition of the present invention may be water-based and forms an aqueous solution. An oil-based carrier solution containing the system of the present invention is an alternative to the aqueous carrier solution. Either aqueous or oil-based solutions further contain thickening agents to provide the (pharmaceutical) composition with the viscosity of a liniment, cream, ointment, gel, or the like. Suitable thickening agents are well known to those skilled in the art.

Pharmaceutically acceptable carriers according to the present invention include, by the way of illustration and not limitation, diluent, disintegrants, binding agents, adhesives, wetting agents, polymers, lubricants, gliands, substances added to mask or counteract a disagreeable texture, taste or odor, flavors, dyes, fragrances, and substances added to improve appearance of the composition. Acceptable carriers include lactose, sucrose, starch powder, maize starch or derivatives thereof, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, saline, dextrose, mannitol, lactose, lecithin, albumin, sodium glutamate, cysteine hydrochloride, and the like. Examples of suitable carriers for soft gelatin capsules include vegetable oils, waxes, fats, semisolid and liquid polyols. Suitable carriers for the preparation of solutions and syrups include, without limitation, water, polyols, sucrose, invert sugar and glucose. Suitable carriers for injectable solutions include, without limitation, water, alcohols, polyols, glycerol, and vegetable oils. The pharmaceutical compositions can additionally include preservatives, solubilizers, stabilizers, wetting agents, emulsifiers, sweeteners, colorants, flavorings, buffers, coating agents, or antioxidants. Suitable pharmaceutical carriers are described in Remington's Pharmaceutical Sciences, Mack Publishing Company, a standard reference text in this field.

Further, the carriers of the (pharmaceutical) composition may also refer to diluents such as, e.g. water, saline, glycerol, ethanol, bacteriostatic water for injection (BWFI), Ringer's solution, dextrose solution, or aqueous solutions of salts and/or buffers etc. Furthermore, substances necessary for formulation purposes may be comprised in said compositions as acceptable carriers such as emulsifying agents, stabilizing agent, surfactants and/or pH buffering substances known to a person skilled in the art.

Said stabilizing agent/stabilizer may act as a tonicity modifier. The term “stabilizing agent” refers to an agent that improves or otherwise enhances stability of the formulation, in particular of the system comprised in said (pharmaceutical) composition. A stabilizing agent which is a tonicity modifier may be a non-reducing sugar, a sugar alcohol or a combination thereof. The tonicity modifiers of the (pharmaceutical) compositions of the present invention ensure that the tonicity, i.e., osmolarity, of the solution is essentially the same as normal physiological fluids and may thus prevent post-administration swelling or rapid absorption of the composition because of differential ion concentrations between the composition and physiological fluids. Preferably, the stabilizing agent/tonicity modifier is one or more of non-reducing sugars, such as sucrose or trehalose or one or more of sugar alcohols, such as mannitol or sorbitol, also combinations of non-reducing sugars and sugar alcohols are preferred.

In (pharmaceutical) compositions of the present invention, the addition of surfactants can be useful to reduce protein degradation during storage. The polysorbates 20 and 80 (Tween 20 and Tween 80) are well established excipients for this purpose. Persons having ordinary skill in the art will understand that the combining of the various components to be included in the formulation can be done in any appropriate order. It is also to be understood by one of ordinary skill in the art that some of these chemicals can be incompatible in certain combinations, and accordingly, are easily substituted with different chemicals that have similar properties but are compatible in the relevant mixture.

The term “buffering agent” as used herein, includes those agents that maintain the pH in a desired range. A buffer is an aqueous solution consisting of a mixture of a weak acid and its conjugate base or a weak base and its conjugated acid. It has the property that the pH of the solution changes very little when a small amount of a strong acid or base is added. Buffer solutions are used as a means of keeping pH at a nearly constant value in a wide variety of chemical applications. In general, a buffer when applied in the formulation of the invention preferably stabilizes the system comprised in said (pharmaceutical) composition of the present invention.

Also, the pharmaceutical composition may comprise one or more adjuvants. The term “adjuvant” is used according to its well-known meaning in connection with pharmaceutical compositions. Specifically, an adjuvant is an immunological agent that modifies, preferably enhances, the effect of such composition while having few, if any, desired immunogenic effects on the immune system when given per se. Suitable adjuvants can be inorganic adjuvants such as, e.g., aluminium salts (e.g., aluminium phosphate, aluminium hydroxide), monophosphoryl lipid A, or organic adjuvants such as squalene or oil-based adjuvants, as well as virosomes.

Said (pharmaceutical) composition of the present invention may be a liquid, preferably aqueous, composition. Further comprised herein is a dried or frozen form of the (pharmaceutical) composition as defined herein. In this context, a frozen form may refer to a composition of at least about −20° C., including such as at least about −30° C., at least about −40° C., at least about −50° C., at least about −60° C., at least about −70° C., at least about −80° C. or more. Preferably, said (pharmaceutical) composition is a liquid, even more preferably aqueous, composition. Thus, said (pharmaceutical) composition may be stored directly in liquid form for later use, stored in a frozen state and thawed prior to use, or prepared in dried form, such as a lyophilized, air-dried, or spray-dried form, for later reconstitution into a liquid form or other form prior to use. Thus, it is envisaged that a (pharmaceutical) composition described herein may be stored by any method known to one of skill in the art. Non-limiting examples include cooling, freezing, lyophilizing, and spray drying the formulation, wherein storage by cooling is preferred.

### In Vivo Therapeutic Applications

The present invention further refers to the composition as defined elsewhere herein for use as a medicament. Hence, the composition of the present invention can also be used for therapy, i.e. the treatment of a viral and/or bacterial disease in a subject in need thereof as defined herein for the diagnostic application of the composition of the present application. Accordingly, the composition of the present invention is particularly suitable for use in a method of preventing or treating a viral and/or bacterial disease in a subject in need thereof.

Hence, the present invention also provides for a method for the prevention or treatment of a viral and/or bacterial disease in a subject, the method comprising administering a therapeutically effective amount of the composition of the present invention to a subject in need thereof.

As such the term “treat”, “treating” or “treatment” as used herein means to reduce (slow down (lessen)), stabilize or inhibit or at least partially alleviate or abrogate the progression of the symptoms associated with the respective disease. Thus, it includes the administration of said composition comprising said system, preferably in the form of a medicament, to a subject, defined elsewhere herein. Those in need of treatment include those already suffering from the disease, here a viral and/or a bacterial disease as described elsewhere herein. Preferably, a treatment reduces (slows down (lessens)), stabilizes, or inhibits or at least partially alleviates or abrogates progression of a symptom that is associated with the presence and/or progression of a disease or pathological condition. “Treat”, “treating”, or “treatment” refers to a therapeutic treatment. In particular, in the context of the present invention, treating or treatment refers to an improvement of the symptom that is associated with said viral and/or bacterial disease as defined elsewhere herein in a subject in need thereof. In this context, the term “treat”, “treating” or “treatment” refers to an antiviral and/or antibacterial therapy that directly attacks the target (viral and/or bacterial) RNA molecules sequences of the specific viruses/bacteria as defined herein. Thus, the composition comprising said system as defined herein may also be used as anti-viral and/or anti-bacterial therapeutic.

The term “prevent”, “preventing”, “prevention” as used herein refers to prophylactic or preventative measures, wherein the subject is to prevent an abnormal, including pathologic, condition in the organism which would then lead to the defined disease, namely said viral and/or said bacterial disease as defined herein. In other words, said terms refer to a medical procedure whose purpose is to prevent a disease meaning inhibiting that a subject will likely suffer from any future viral and/or bacterial disease as defined herein. As used herein, such terms also refer to the reduction in the risk of acquiring or developing a given condition in a patient diagnosed with any viral and/or bacterial disease as defined herein. Thus, it also includes the administration of said composition comprising said system, preferably in the form of a medicament, to a subject, defined elsewhere herein. Those in need of the prevention include those prone to having the disease, such as said viral and/or said bacterial disease as defined herein. In other words, those who are of a risk to develop such disease and will thus probably suffer from said disease in the near future. Thus, the composition as defined herein may also be used as a prophylaxis, not as a therapeutic agent for a subject as defined herein that has already been infected. This may prevent Cas13 protein from being overwhelmed by the viral RNA molecules.

The term “subject” when used herein includes mammalian and non-mammalian subjects. Preferably the subject of the present invention is a mammal, including human, domestic and farm animals, non-human primates, and any other animal that has mammary tissue. In some embodiment the mammal is a mouse. In some embodiment the mammal is a rat. In some embodiment the mammal is a guinea pig. In some embodiment the mammal is a rabbit. In some embodiment the mammal is a cat. In some embodiment the mammal is a dog. In some embodiment the mammal is a monkey. In some embodiment the mammal is a horse. In a most preferred embodiment the mammal of the present invention is a human. A subject also includes human and veterinary patients.

The uses, kits and compositions described in this document are generally applicable to both human and veterinary diseases. Where the subject is a living human who may receive treatment for a disease or condition as described herein, it is also addressed as a “patient”. In some embodiments the subject of the present invention is of a risk to develop said viral and/or said bacterial disease as described herein. In some embodiments the subject of the present invention suffers from said viral and/or said bacterial disease as described herein. The term “suffering” as used herein means that the subject is not any more a healthy subject. The term “healthy” means that the respective subject has no obvious or noticeable hallmarks or symptoms of the respective disease. This further means that the subject suffering from said viral and/or said bacterial disease is a subject “in need” of the respective treatment with the composition comprising said system of the present invention. Those in need of treatment include those already suffering from the disease as well as those prone to having the disorder or those in whom the disorder is to be prevented (prophylaxis).

The composition comprising said system of the present invention for use in a method of preventing or treating a subject suffering from a viral and/or bacterial disease as defined elsewhere herein are generally administered to the subject in a therapeutically effective amount. Said therapeutically effective amount is sufficient to inhibit or alleviate the symptoms of said viral and/or said bacterial disease. By “therapeutic effect” or “therapeutically effective” is meant that the conjugate for use will elicit the biological or medical response of a tissue, system, animal or human that is being sought by the researcher, veterinarian, medical doctor or other clinician. The term “therapeutically effective” further refers to the inhibition of factors causing or contributing to the disease or disorder. The term “therapeutically effective amount” includes that the amount of the agent when administered is sufficient to significantly improve the progression of the disease being treated or to prevent development of said disease. According to a preferred embodiment, the therapeutic effective amount is sufficient to alleviate or heal said viral and/or bacterial disease as defined herein.

The therapeutically effective amount will vary depending on said composition of the present invention, the disease and its severity and on individual factor of the subject and/or also how the administration (also called the delivery) of said works. Therefore, the composition of the present invention will not in all cases turn out to be therapeutically effective, because the method disclosed herein cannot provide a 100% safe prediction whether or not a subject may be responsive to the detection system, since individual factors are involved as well. It is to expect that age, body weight, general health, sex, diet, drug interaction and the like may have a general influence as to whether or not the compound for use in the treatment of a subject suffering from said disease will be therapeutically effective.

The term “administering” or “administered” or “administration” used throughout various aspects of the present invention means that the composition comprising said system as defined herein are given to the respective subject in an appropriate form and dose and using appropriate measures. The administration of the composition according to the present invention can be carried out by any method known in the art.

The administration of said composition or said composition in a therapeutically effective amount as defined elsewhere herein may be performed by inhalation, injection, infusion, or orally. The administration of the composition as defined elsewhere herein or the composition comprising said composition may be performed intraperitoneally, intravenously, intraarterially, subcutaneously, intramuscularly, parenterally, transdermally, intraluminally, intrathecally and/or intranasally or directly into tissue.

Where said composition is to be administered by infusion, it can be dispensed with an infusion bottle containing sterile pharmaceutical grade water or saline. Where said composition is administered by injection, an ampoule of sterile water for injection or saline can be provided so that the ingredients may be mixed prior to administration. Where the composition is to be administered by inhalation, adequate inhalation devices may be used known to a person skilled.

In a particular embodiment, said administration of said composition may comprise the delivery to said subject via different methods based on the fact whether said composition is delivered as a DNA, RNA or protein based system. If it is delivered as a DNA based system—in other words when said composition may be administered as defined herein as a nucleic acid molecule comprising a nucleotide sequence encoding said Cas13 protein as defined herein and a nucleotide sequence encoding said gRNA as defined herein, wherein said nucleic acid molecule is DNA—it may be administered using any vector as described herein such as adeno-associated virus (AAV) vector for AAV-mediated gene delivery or adenoviral or lentiviral vectors optimized for expression of said at least one gRNA and said at least one Cas13 gene and/or using particles and/or nanoparticles. If it is delivered as a RNA based system—in other words when said composition may be administered as defined herein as a nucleic acid molecule comprising a nucleotide sequence encoding said Cas13 protein as defined herein and a nucleotide sequence encoding said gRNA as defined herein, wherein said nucleic acid molecule is RNA—it may be administered using particular particles and/or nanoparticles and/or liposomes and/or exosomes comprising said composition as it is known to a person skilled in the art, preferably via nanoparticles. If it is delivered as a protein based system—in other words when said composition may be administered as defined herein comprising said at least one Cas13 protein as defined elsewhere herein and said at least one gRNA as defined herein—it may also be administered using particular particles and/or nanoparticles and/or liposomes and/or exosomes comprising said composition as it is known to a person skilled in the art, preferably via nanoparticles. For the RNA and protein based system, said at least one gRNA may have already been chemically synthesized in correct form before said delivery of said composition, whereas for the DNA based system, said gRNA may need to be transcribed in the cells.

The viral disease, which is prevented or treated by the use of said composition may refer to a disease caused by a DNA virus, a RNA virus, or a retrovirus as defined elsewhere herein, preferably caused by a RNA virus. In specific embodiments, the viral infection is caused by a double-stranded RNA virus, a positive sense RNA virus, a negative sense RNA virus or a combination thereof. In a more preferred embodiment, the viral disease, which is prevented or treated by the use of said composition is any one of a coronavirus disease, influenza A, ebola, measles, hepatitis C, tick-borne encephalitis (TBE), Venezuelan Equine Encephalitis (VEE) viral infection, dengue fever, yellow fever, or zika fever, even more preferably coronavirus or influenza disease. The “coronavirus” may be SARS-CoV, SARS-CoV2 or MERS or a related new zoonotic or mutant coronavirus. The Coronavirus group consists of enveloped positive stranded RNA viruses belonging to the family Coronaviridae and comprise subtypes referred to as Alpha-, Beta-, Gamma- and Delta coronavirus. Alpha and Beta affect mammals, while Gamma affects birds and Delta can affect both. The coronavirus family comprises several well-known disease-causing members. The Betacoronavirus family, has so far posed the biggest risk to humans and now includes the most well-known virus targets including Severe Acute Respiratory Syndrome coronavirus (SARS-CoV-1), Middle East Respiratory Syndrome coronavirus (MERS-CoV), and the newest form to emerge, the novel coronavirus, (SARS-CoV-2). In a most preferred embodiment, said viral disease, which is prevented or treated by the use of said composition comprising said system, is the COVID-19 disease. In the case of COVID-19, the clinical spectrum of SARS-CoV-2 infection appears to be wide, encompassing asymptomatic infection, mild upper respiratory tract illness, and severe viral pneumonia with respiratory failure and even death, with many patients being hospitalized.

Since any bacterium also comprises ssRNA, which can be targeted/detected and then also cleaved/degraded, thus inactivating said bacterial ssRNA, said therapeutic approach using said composition according to the present can also be applied for treating or preventing any bacterial disease caused by a bacterium such as a drug resistant bacterium as defined elsewhere herein.

In alternative embodiments, the composition for the use in the treatment of said viral and/or bacterial disease of the present invention, may also be administered in combination with an additional therapeutic agent (drug). Drugs or therapeutic agents useful in this regard include without limitation drug-like molecules, proteins, peptides, and small molecules. Protein therapeutic agents include, without limitation peptides, enzymes, antibodies, structural proteins, receptors and other cellular or circulating proteins as well as fragments and derivatives thereof, preferably an additional therapeutic agent/drug in the context of the present invention may be a drug for the use in viral and/or bacterial diseases as described elsewhere herein, especially for combinatorial therapy in said diseases. Said combination according to the present invention can be administered as a combined formulation or separate from each other.

Also comprised by the present invention is a method of preventing or treating a viral or a bacterial disease in a subject, the method comprising administering to the subject a therapeutically effective amount of the composition as defined herein. Also comprised herein is the use of said composition as defined elsewhere herein for the manufacture of a medicament for therapeutic application in a viral or a bacterial disease in a subject. The definitions and embodiments made with regard to the first and second medical uses may be applied in this context as well.

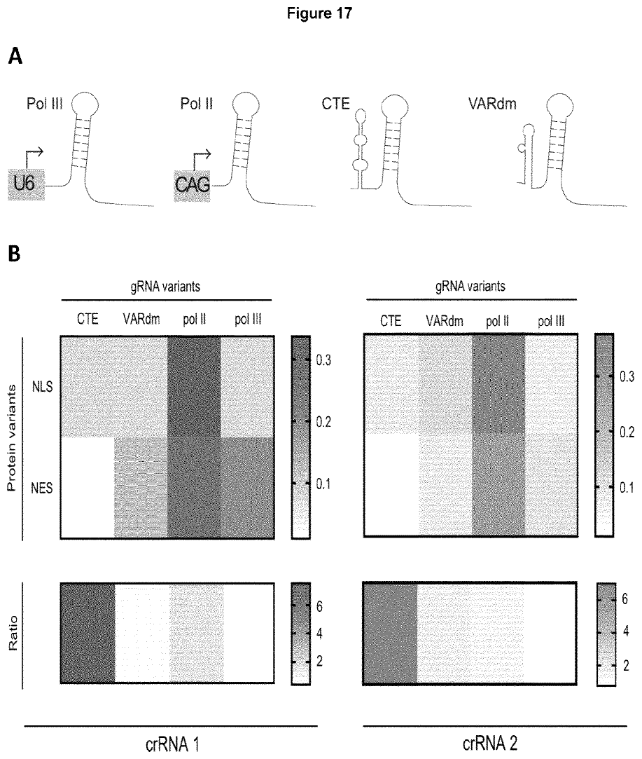

As mentioned elsewhere herein a composition comprising a CRISPR system comprising i) at least one Cas13 protein or a nucleotide sequence encoding said Cas13 protein fused with at least one NLS or with at least one NES and ii) at least one gRNA or a nucleotide sequence encoding said gRNA capable of hybridizing with one or more target RNA molecules, which is fused to at least one viral export element is also comprised by the present invention, wherein such RNA export elements, such as CTE or VARdm, drive the export of gRNAs from the nucleus to the cytosol. Here, the nucleotide sequence encoding gRNA, preferably wherein the nucleotide sequence is DNA, is fused with at least one viral export element. Said at least one viral export element as defined herein is fused as defined elsewhere herein to the 5′ end of the nucleotide sequence encoding said gRNA, preferably wherein said nucleotide sequence is DNA. In this context, fusing means again that the nucleotide sequence encoding said gRNA and the at least one viral export element are fused via a linker such as any nucleotide linker. Thus, a composition is also comprised herein, wherein the DNA nucleotide sequence encoding said gRNA of the abovementioned composition (comprising at least one Cas13 protein or a nucleotide sequence encoding said protein fused with at least one NLS or with at least one NES) is fused via linker such as any nucleotide linker with at least one viral export element as defined herein at the 5′ end of said nucleotide sequence. Again, the term “at least one viral export element” refers to two, three, four, five or more viral export elements as defined herein. In a preferred embodiment, said at least one viral export element is a CTE or a VARdm, preferably a CTE, which is fused as defined herein with the nucleotide sequence encoding said gRNA, which is preferably a DNA sequence. A fusion of the nucleotide sequence encoding said gRNA with at least one viral export element as defined herein proves to be advantageous, since it increases the knockdown efficiency of said Cas13 protein fused to NLS or NES, preferably when fusing a viral CTE to said gRNA, most preferably when fusing a viral CTE to said gRNA and fusing a NES to said Cas13 protein (see FIG. 17). Such fusion to the gRNA can also be applied on the protein level of such composition.

Any definitions mentioned above concerning the composition comprising inter alia Cas13 protein fused with at least one NLS fused with at least one NES can be applied (in any combination), where applicable to the composition comprising inter alia Cas13 protein fused with at least one NLS or with at least one NES also comprising at least one viral export element fusion to said gRNA.

### Nucleic Acid Molecules

Further comprised by the present invention is a nucleic acid molecule comprising a nucleotide sequence encoding said Cas13 protein and/or a nucleotide sequence encoding said gRNA as defined elsewhere herein. This means that when such nucleic acid molecule is applied comprising a nucleotide sequence encoding said Cas13 protein, said Cas13 protein as defined herein which is fused to said at least one NLS fused to said at least one NES (which are also encoded by nucleotide sequences) as described according to the present invention, is encoded by said molecule. Further, when said nucleic acid molecule is applied comprising a nucleotide sequence encoding said gRNA, said gRNA as it is defined herein, e.g. with regard to said modifications may be encoded by said molecule.

Nucleic acid molecules comprising a nucleotide sequence encoding said Cas13 protein and/or said gRNA include DNA, such as cDNA or genomic DNA, and RNA. Preferably, embodiments reciting “RNA” are directed to mRNA.

The present invention also relates to nucleic acid molecules as defined herein comprising nucleotide sequences encoding for said Cas13 protein and/or said gRNA as described herein. Since the degeneracy of the genetic code permits substitutions of certain codons by other codons specifying the same amino acid, the invention is not limited to a specific nucleic acid molecule encoding said Cas13 and/or gRNA as defined herein but includes all nucleic acid molecules comprising nucleotide sequences encoding a functional Cas13 protein and/or gRNA as defined herein.

In some embodiments, a nucleic acid molecule comprising a nucleotide sequence encoding said Cas13 protein and/or said gRNA disclosed in this application, such as DNA or RNA, may comprise nucleotide sequences which are “operably linked” to one another, i.e as described above the nucleotide sequence encoding said at least one Cas13 protein as described elsewhere herein (if more than one Cas13 protein is used, also more than one nucleotide sequences encoding said Cas13 proteins may be applied), a nucleotide sequence encoding said at least one gRNA as defined elsewhere herein (if more than one gRNA is used, also more than one nucleotide sequences encoding said gRNAs may be applied), and a nucleotide sequence encoding said localization signal as defined elsewhere herein. Said nucleotide sequences are operably linked to one another. In this regard, an operable linkage is a linkage in which the sequence elements of one nucleotide sequences and the sequence elements of another nucleotide sequences are connected in a way that enables expression of the detection system.

The invention also includes nucleic acid molecules as described elsewhere herein, which include additional mutations outside the indicated sequence positions of experimental mutagenesis. Such mutations are often tolerated or can even prove to be advantageous, for example if they contribute to an improved folding efficiency, serum stability, thermal stability or ligand binding affinity.

A nucleic acid molecule disclosed in this application may be “operably linked” to a regulatory sequence (or regulatory sequences) to allow expression of this nucleic acid molecule.

A nucleic acid molecule, such as DNA or RNA, is referred to as “capable of expressing a nucleic acid molecule” or capable “to allow expression of a nucleotide sequence” if it includes sequence elements which contain information regarding to transcriptional and/or translational regulation, and such sequences are “operably linked” to the nucleotide sequence encoding said Cas13 protein and/or said gRNA as defined elsewhere herein. An operable linkage is a linkage in which the regulatory sequence elements and the sequence to be expressed are connected in a way that enables gene expression. The precise nature of the regulatory regions necessary for gene expression may vary among species, but in general these regions include a promoter which, in prokaryotes, contains both the promoter per se, i.e. DNA elements directing the initiation of transcription, as well as DNA elements which, when transcribed into RNA, will signal the initiation of translation. Such promoter regions normally include 5′ non-coding sequences involved in initiation of transcription and translation, such as the −35/−10 boxes and the Shine-Dalgarno element in prokaryotes or the TATA box, CAAT sequences, and 5′-capping elements in eukaryotes. These regions can also include enhancer or repressor elements as well as translated signal and leader sequences for targeting the native polypeptide to a specific compartment of a host cell.

In addition, the 3′ non-coding sequences may contain regulatory elements involved in transcriptional termination, polyadenylation or the like. If, however, these termination sequences are not satisfactory functional in a particular host cell, then they may be substituted with signals functional in that cell.

Therefore, a nucleic acid molecule of the present invention can include a regulatory sequence, such as a promoter sequence. In some embodiments a nucleic acid molecule of the present invention includes a promoter sequence and a transcriptional termination sequence. Suitable promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the (3-actin promoter, the phosphoglycerol kinase (PGK) promoter, the EF1α promoter and U6 promoter, TetOn/Off, 7SK, CAG, CBh, UbC or any tetracycline-dependent or interferon-dependent promoter.

### Vector

The nucleic acid molecules of the present invention can also be part of a vector or any other kind of cloning vehicle, such as a plasmid, a phagemid, a phage, a baculovirus, a cosmid or an artificial chromosome, preferably part of a vector.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleotide sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words, samples comprising a masking construct may be delivered to a cell, for example in a suitable delivery vesicle and if the target is present in the delivery vesicle the CRISPR effector is activated and a detectable signal generated. The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) encoding sequences, such as 1-2, 1-3, 1-4 1-5, 3-6, 3-7, 3-8, 3-9, 3-10, 3-8, 3-16, 3-30, 3-32, 3-48, 3-50 RNA(s). In a single vector there can be a promoter for each RNA advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ′4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector, is to express an array of promoter-RNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjournals.org/content/34/7/e53.short and nature.com/mt/journal/v16/n9/abs/mt2008144a.html). In an embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters-especially as to the numbers of RNAs or guides discussed herein, without any undue experimentation.

Such cloning vehicles can include, aside from the regulatory sequences described above and a nucleic acid molecule comprising a nucleotide sequence encoding said Cas13 protein and/or gRNA as described herein, replication and control sequences derived from a species compatible with the host cell that is used for expression as well as selection markers conferring a selectable phenotype on transformed or transfected cells. Large numbers of suitable cloning vectors are known in the art and are commercially available.

The nucleic acid molecule as described herein and in particular a cloning vector containing the coding sequence of such Cas13 protein and/or gRNA as defined herein can be transformed into a host cell capable of expressing the gene. Transformation can be performed using standard techniques (Sambrook, J. et al. (1988) Molecular Cloning: A Laboratory Manual, 2nd Ed).

### Host Cell

Thus, the present invention is also directed to a host cell comprising said nucleic acid molecule or said vector as described herein.

The transformed host cells are cultured under conditions suitable for expression of the nucleotide sequences encoding said Cas13 protein and/or said gRNA as defined herein. Suitable host cells can be prokaryotic, such as Escherichia coli (E. coil) or Bacillus subtilis, or eukaryotic, such as Saccharomyces cerevisiae, Pichia pastoris, SF9 or High5 insect cells, immortalized mammalian cell lines such as HeLa cells or CHO cells or primary mammalian cells, preferably E. coli.

### Kit

The present invention also relates to a kit comprising the composition as defined herein by the present invention. Thus, when a kit comprises the composition, said composition may be provided in a vial or a container, preferably also comprising in said vial or container at least one carrier as defined herein. Thus, when a kit comprises the pharmaceutical composition, said composition may be provided in a vial or a container, preferably also comprising at least one pharmaceutically acceptable carrier and/or an adjuvant as defined herein. Further, said kits may be associated with a notice in the form prescribed by a governmental agency regulating the manufacture, use or sale of pharmaceuticals or biological products, reflecting approval by the agency of the manufacture, use or sale of the product for human administration or diagnostics. Said kits may comprise the composition as defined herein, preferably in a vial or container, in dried form, such as a lyophilized, air-dried, or spray-dried form (in form of a powder), for later reconstitution into a liquid form or other form prior to use. Further, said kits may also comprise the composition, preferably in a vial or container, in a frozen state, being thawed prior to use.

The kits according to the present invention may also comprise a delivery system. Delivery systems for transgenes are well known in the art. By means of example, the Cas transgene and thus the composition comprising said CRISPR system in the form of DNA may be delivered in for instance eukaryotic cells by means of a vector (e.g., AAV, adenovirus, lentivirus, plasmid) and/or by means of a particle and/or a nanoparticle as defined elsewhere herein. The composition comprising said CRISPR system in the form of RNA or on the protein level may be delivered in for instance eukaryotic cells by means of particles and/or nanoparticles and/or liposomes and/or exosomes as also described elsewhere herein. Such delivery systems may also be comprised in the one or more vials or containers of the kits as defined above or in additional one or more vials or containers of said kit, preferably further comprising in said one or more vials or containers any excipient suitable for said delivery system to be mixed with/contacted with.

The kit according to the present invention may also comprise a label. A label as used herein may refer to a compound capable of targeting said system comprised in said composition. Said compound may refer to an antibody, beads coupled/coated to an antibody f.e. dynabeads coupled/coated to an antibody, preferably which is used for classical purification processes such as chromatography when the composition may be produced on the protein level. Further, said label can be oligodT magnetic beads. Such beads may be capable of binding to the polyA tail of the full-length RNA, when the composition on the RNA level is produced and then further purified as known to the person skilled in the art.

In some embodiments, said label may also be comprised in the one or more containers or vials of the kit as defined above comprising said composition or in additional one or more vials or containers of said kit, preferably further comprising in said one or more vials or containers any excipient suitable for said label to be mixed with/contacted with.

### In Vitro and In Vivo Applications

The present invention also relates to a method of producing the composition of the present invention, in other words producing said CRISPR system comprised by said composition. In this context, the composition is produced starting from the nucleic acid coding for said Cas13 protein and/or said gRNA as defined herein by means of genetic engineering methods. Again, in this context, said Cas13 protein that is encoded is a Cas13 protein fused to said at least one NLS which is fused to at least one NES as described throughout the present invention. Thus, the end product of the provided production method is the composition of the present invention, either a composition comprising said system in form of a nucleic acid molecule comprising a nucleotide sequence encoding said Cas13 protein and/or said gRNA as defined elsewhere herein, wherein said nucleic acid molecule is DNA (“DNA based system”) or RNA (“RNA based system”) or in the form of a protein-based system.

Thus, when the term “nucleic acid coding by means of genetic engineering” is used herein with regard to the DNA based system, it refers to the in vitro usage of artificial gene synthesis (for said nucleotide sequence encoding said Cas13 protein and/or gRNA). Such synthesis refers to a method that is used in synthetic biology to construct and assemble genes from nucleotides de novo as it is known to a person skilled in the art. When the term “nucleic acid coding by means of genetic engineering” is used herein with regard to the RNA based system, it refers to the in vitro usage of RNA synthesis (for said Cas13 mRNA and/or said gRNA) as known to a person skilled in the art. When the term “nucleic acid coding by means of genetic engineering” is used herein with regard to the protein based system, it should be divided for carrying out said method in vivo or in vitro. In some embodiments and when referred to the protein based system of said composition as defined elsewhere herein, the method can be carried out in vivo, meaning that the composition can, for example, be produced in a bacterial or eukaryotic host organism and then isolated from this host organism or its culture by means known to the person skilled in the art. When producing the composition in vivo, a nucleic acid molecule comprising a nucleotide sequence encoding said Cas13 protein and/or said gRNA as defined herein is introduced into a suitable bacterial or eukaryotic host organism by means of recombinant DNA technology. For this purpose, the host cell is first transformed with a cloning vector that includes a nucleic acid molecule as described herein using established standard methods. The host cell is then cultured under conditions, which allow expression of the heterologous DNA and thus the synthesis of the corresponding CRISPR system. Subsequently, the composition is then isolated either from the host cell or from the cultivation medium. In other embodiments and still when referred to the protein based system, the nucleic acid coding of said method can be carried out in vitro, meaning that it is also possible to produce said composition of the present invention by use of an in vitro translation system.

After the nucleic acid coding as defined above, the method may further comprise the obtaining of the produced composition.

When the composition is produced on the DNA level (meaning nucleotide sequence encoding said Cas13 protein and/or gRNA), the term “obtain” or “obtaining” when used in this respect can be understood in that the produced composition is extracted from the used (reaction) buffer after the nucleic acid coding by using f.e. artificial gene synthesis and potentially purified before said composition can be used. Such classical purification process of a DNA based system may comprise the usage of silica columns as known to the person skilled in the art. When the composition is produced on the RNA level (meaning Cas13 mRNA and/or gRNA), the term “obtain” or “obtaining” when used in this respect can be understood in that the produced composition is extracted from the used (reaction) buffer after the nucleic acid coding by using f.e. RNA synthesis and potentially purified before said composition can be used. Such classical purification process of a RNA based system may comprise the usage of oligodT magnetic beads as known to the person skilled in the art. Such beads are capable of binding to the polyA tail of the RNAs of the components of said CRISPR system, wherein by-products may be separated, thus achieving better purity of said components When the composition is produced on the protein level in vivo (Cas13 protein and/or gRNA), the term “obtain” or “obtaining” when used in this respect can be understood in that the produced composition is then isolated from the host organism or its culture by means known to the person skilled in the art as described above. When the composition is produced on the protein level in vitro, the term “obtain” or “obtaining” can be understood in that the produced composition is extracted from the used (reaction) buffer after the nucleic acid coding by using f.e. an in vitro translation system and potentially purified before said composition can be used. A classical purification process which may be used may refer to suitable chromatography as known to a skilled artisan. The term “recover” or “recovering” can be used interchangeably with the term “obtain” or “obtaining” herein.

The abovementioned paragraphs and definitions concerning the nucleic acid molecule, vector, host cell, kit and in vitro as well as in vivo application can also be applied, where applicable, to the composition comprising a clustered, regularly interspaced, short palindromic repeats (CRISPR) system comprising i) at least one CRISPR-associated protein 13 (Cas13) or a nucleotide sequence encoding said Cas13 protein fused with at least one nuclear localization signal (NLS) or with at least one nuclear export sequence (NES) and ii) at least one guide RNA (gRNA) or a nucleotide sequence encoding said gRNA capable of hybridizing with one or more target RNA molecules, which is fused to at least one viral export element as defined elsewhere herein.

### EXAMPLES OF THE INVENTION

### Materials and Methods

Cloning of Key Constructs.

Cas13 coding sequences were ordered as synthetic fragment (gBlock, IDT) and cloned into a pCAG backbone. Different localization sequences were fused to the C-terminus via ligation or Gibson assembly. Cas13 gRNA scaffold sequences were ordered as synthetic fragment (gBlock, IDT) and cloned into a U6 backbone. Different gRNAs were cloned into the scaffold by annealing complementary oligonucleotides (Metabion). HA-tagged SARS-CoV-2 was amplified by One-step RT-PCR (SuperScript IV One-step RT-PCR, Thermo Fisher) from a fragmented, synthetic SARS-CoV-2 genome (Twist Bioscience). All Luciferases used here were fused to a C-terminal Degron derived from mouse ornithine decarboxylase to closely couple the RNA and protein level. tRNAs and ribozymes were ordered as synthetic fragment (gBlock, IDT) and added to the 3′ end of the gRNA via Gibson assembly. 5′ and 3′ beta globin stabilizing UTRs were amplified from HEK293T genomic DNA and cloned up- or downstream of Cas13 coding sequences.

Standard RNA Preparation.

Templates for in vitro transcription were generated by linearization of plasmid templates. Digests were purified (Monarch DNA Cleanup Kit, New England Biolabs) and subsequently used for mRNA synthesis (HiScribe T7 ARCA mRNA with tailing Kit, New England Biolabs). Synthesized RNA was purified (Monarch RNA Cleanup, New England Biolabs) and stored at −80° C. gRNA templates were generated by extension of partially overlapping oligonucleotides (Q5 Mastermix, New England Biolabs) and directly used for in vitro transcription. Synthesized gRNAs were purified (Monarch RNA Cleanup, New England Biolabs) and stored at −80° C.

Cell Lines and Cultivation.

All experiments were performed in HEK293T cells. Cells were maintained at 37° C., in 7.5% CO₂, H₂O saturated atmosphere. Advanced DMEM medium with 10% FBS was used for maintenance.

Knockdown Efficiencies Measured Via Luciferase Assays.

Cells were transfected with different Cas13 systems together with either mRNA or DNA coding for Nanoluciferase. 24-72 h after the transfection cells were lysed and Nanoluciferase was measured (NanoGlo Assay, Promega). In experiments where the on-target and off-target activity was quantified, Nanoluciferase along with Firefly Luciferase was transfected in the same well. 24-72 h after the transfection cells were lysed and both luciferases were measured independently (NanoGlo Dual Luciferase Assay, Promega).

Assessment of Protein Stability and SARS-CoV-2 RdRP Knockdown.

72 h after the transfection with different Cas13 variants, cells were lysed in M-PER buffer (Thermo Fisher) supplemented with Halt Protease Inhibitor Cocktail (Thermo Fisher). Lysates were prepared in Laemmli Buffer (Sigma-Aldrich) and run on a SDS PAGE (Thermo Fisher). Proteins were transferred to a PVDF membrane overnight at 20V, blocked, incubated with either primary M2 Anti-FLAG (Merck Millipore) antibody to stain for Cas13 or HA antibody (Cell Signaling) for HA-tagged SARS-CoV-2 RdRP and imaged with Amersham ECL Prime substrate (Sigma-Aldrich).

Cas13 Protein Localization.

72 h after the transfection with different Cas13 variants cells were fixed in formalin, permeabilized and incubated with primary M2 Anti-FLAG (Merck Millipore) antibody. After incubation with secondary antibody cells were additionally DAPI stained and imaged at an EVOS Cell Imaging System (Thermo Fisher).

SARS-CoV-2 Inhibition.

Cells were transfected with mRNA coding for ACE2 (20 ng), Cas13 NLS-NES (40 ng) and gRNA (40 ng) in 96 well plates. mRNA transfection was carried out with MessengerMax (Thermo Fisher). 24 h after the transfection cells were infected with SARS-CoV-2-GFP under BSL3 conditions. 72 h after the transfection cells were lysed and the virus was inactivated by heating at 60° C. in Trizol. RNA was extracted by Trizol/Chloroform extraction. Extracted RNA was then quantified via RT-qPCR (Luna Universal Probe One-Step Assay, New England Biolabs) in a CDC approved diagnostic assay (for viral N gene, IDT).

Measurement of Cellular Translation.

Cells were transfected with Cas13, mRuby3 and a gRNA targeting mRuby3. 72 h after the transfection nascent protein translation was recorded and stained according to manufacturer's protocol (Protein Synthesis Assay Kit, Cayman Chemical). Stained cells were analyzed via FACS.

Ribosome Assay.

Cells were transfected with Cas13, gRNA and target RNA. 72 h after the transfection total RNA was extracted (Monarch Total RNA Miniprep Kit, New England Biolabs) and analyzed on a agarose gel. In comparison, purified 80S ribosomes were incubated for 1 h in NEB 4 buffer with purified Cas13 protein (generated via in vitro translation: NEBExpress, New England Biolabs), in vitro transcribed gRNA and in vitro transcribed target RNA (as described before). The reaction was quenched via the addition of 0.5 M EDTA. The reaction was then incubated for 1 h at 37° C. with Proteinase K and subsequently analyzed on an agarose gel.

Optimization of RNA Delivery.

To stabilize the Cas13 mRNA, a fragment of the 5′ beta globin UTR and two copies of the beta globin 3′UTR were added to the Cas13 coding sequences. Additionally, a genetically encoded 162 nucleotides polyA tail was added. UTP was replaced by N1-Methylpseudo-UTP (Jena Bioscience) and 5′ capping was performed post-transcriptionally by Vaccina Capping System and 2′-O-Methlytransferase (both New England Biolads). gRNAs were stabilized by replacing the two terminal nucleotides at the 5′ and 3′ end by 2′O-Methyl-Phosphorothioate via chemical synthesis (IDT).

### Results

### Example 1: Characterization of Optimal Knockdown Conditions For Cas13

To find the optimal composition of a Cas13-based knockdown tool, Cas13d variants and miRNA-based RNA interference were directly compared in a Firefly Luciferase knockdown assay. The known Cas13d-NLS with 22 bp gRNA yield a moderate knockdown efficiency, which was improved by increasing the gRNA length to 30 bp and by fusing the Cas13 protein to a NLS and NES together. This optimized system is even more efficient than latest generation RNA interference (FIG. 1).

Originally, the optimal gRNA length for Cas13d was found to be 22 bp in an in vitro assay with purified components (Konermann et al. (2018), Cell 173(3): 665-676). The inventors characterized the knockdown efficiency of different gRNAs lengths in a mammalian cell line, by targeting a co-transfected Nanoluciferase. In contrast to previous in vitro work the optimal gRNA length was in the range of 26 to 30 bp (FIG. 2).

The inventors hypothesized that the addition of a tRNA, which undergoes processing by RNAse P/Z or a self-cleaving ribozyme to the 3′ of the gRNA could support the gRNA folding. Additionally, both motifs cleave off a U-stretch which is added to the gRNA during the transcriptional termination of a Pol III promoters (e.g. U6 promoter). The addition of both motifs showed improved knockdown efficiencies compared to a non-modified gRNA (FIG. 3).

### Example 2: Exploitation of Different Strategies to Transfer Cas13d to the Cytosol

The inventors compared the knockdown efficiency of Cas13d proteins with different localization signals in a luciferase-based assay. Pol III driven gRNAs are expressed and remain in the nucleus, therefore Cas13d exported to the cytosol (NES) is hardly active. Additionally, Western Blot analysis showed that the protein is unstable if it is not bound to a gRNA in the same compartment. A Cas13d variant which is imported to the nucleus (NLS) is active and stable because of being present in the same compartment as the gRNA and forming a gRNA/Cas13d complex. For a Cas13d fusion protein consisting of both localization signals (NLS-NES) a shuffling mechanism was expected. The protein is imported to the nucleus (NLS), picks up the gRNA and then being exported to the cytosol (NES) again, where the majority of the luciferase mRNA is targeted for degradation. This NLS-NES strategy stabilized the protein in the cytosol and maximized the knockdown efficiency. A complementary strategy in which the gRNA itself is exported to the cytosol by expression from a Pol II promoter supports the hypothesis, because in this case, Cas13d-NES is most active. By directly comparing the two strategies, Cas13d-NLS-NES is most efficient (FIG. 4).

### Example 3: Evaluation of Cas13d's Specificity

For all Cas13 systems in vitro experiments suggested a target RNA-based activation mechanism, which unleashes an unspecific RNA degradation, called collateral RNA cleavage (Abudayyeh et al. (2016), Konermann et al. (2018)). Surprisingly, this unspecific RNA degradation is absent in all eukaryotic cell systems (Abudayyeh et al. (2017) Nature 550, 280-284, Konermann et al. (2018)). Transcriptomes of eukaryotic cells are much more complex than transcriptomes of prokaryotes (intron/exon structure, non-coding RNAs, small RNAs etc.). The inventors hypothesized that the differences seen in the Cas13 mechanism between pro- and eukaryotic might directly be related to differences in the activity of the Cas13 system. For a moderately active Cas13 system collateral RNA cleavage might be overseen in a highly complex eukaryotic transcriptome. Therefore, the inventors tested for collateral cleavage of highly active Cas13-IDG. As expected, the know Cas13d system as well as a miRNA did not result in substantial inhibition of a co-transfected and unrelated Nanoluciferase, when Firefly Luciferase was targeted. For Cas13-IDG an increase in Nanoluciferase knockdown was measured along with an increased knockdown of Firefly Luciferase (FIG. 5). This led the inventors to the conclusion that Cas13-IDG exhibits the previously overseen feature of collateral activity which can be harnessed for target-transcript induced viral inhibition.

### Example 4: Efficient Knockdown of Heterologous Expressed mRNAs

The prior art came to very different results with regard to the efficiency of the individual systems, which is why the inventors developed a luciferase-based assay that enabled them to directly compare the known Cas13 systems with several gRNA and target RNAs in a 96-well format (FIG. 6A). It was shown that Cas13d from Ruminococcus flavefaciens XPD3002 is most efficient to degrade the target RNA. Based on this assay, the inventors were able to test numerous modifications to the gRNA and the Cas13 protein and to identify three modifications that increased the knockdown efficiency of Cas13d from 50% to over 90% (FIG. 6B). The optimization steps involved an extension of the gRNA, the fusion of a tRNA to support the folding of the gRNA, and the fusion of a nuclear and additionally a cytoplasmic localization signal to the Cas13d protein. The fusion of both localization signals together led to the localization of Cas13d into the cellular cytoplasm (FIG. 6C). In contrast to the published Cas13d variant, this step for the first time allows to degrade cytosolic instead of nuclear RNAs. The modified Cas13d composition comprising each of the three modifications refers to Cas13-IDG.

In the course of the work on Cas13-IDG, the inventors were able to demonstrate the initially described collateral activity in prokaryotic system of a Cas13 composition in mammalian cells. In contrast to the known collateral activity in vitro (experimental cell-free systems) and bacterial systems, Cas13-IDG seems to have a preference to bind and cut 28S ribosomal RNA at a defined position (FIG. 6D, 6E), which ultimately leads to a transient inhibition of the global cellular protein translation (FIG. 8). By analyzing the impact of purified Cas13 on purified 80S ribosomes in an in vitro system, it was confirmed that the 28S rRNA is cleaved directly by Cas13 and not by an endogenous cellular mechanism (FIG. 9).

### Example 5: Inhibitory Effect of Cas13-IDG on SARS-CoV-2

Based on the general optimization and the discovery of the influence of Cas13-IDG on the cellular translation, the inventors further developed these findings towards an anti-viral therapeutic. To do this, they use gRNAs that are complementary to different areas of the SARS-CoV-2 virus, focusing on the 5′ and 3′ parts of the virus. Since these two areas are present both in the genome of the virus and in all subgenomic mRNAs, the antiviral effect of Cas13 may be strongest at these positions. In addition, it was recently shown that there is massive replication of the viral RNA after infection, which after a short time exceeds the amount of endogenous RNA. The inventors therefore expect a four level viral inhibition, if Cas13 is directed against the 5′ or 3′ viral UTR: 1) Direct targeting of the viral genome 2) Targeting of copies of the viral genome 3) Degradation of viral mRNAs 4) A dramatic activation of Cas13-IDG in infected cells and thus a strong collateral effect on protein translation of the virus (FIG. 7A).

The inventors showed that Cas13-IDG together with a gRNA targeting the artificially expressed RdRp protein of SARS-CoV-2, knocked down the protein by approx. 90% (FIG. 7B). Building on this, the inventors started to test the antiviral effect of Cas13-IDG on SARS-CoV-2 infected cells (under BSL-3 conditions). Initial results showed a virus reduction of up to 80%, depending on the position of the gRNA (FIG. 7C). In this experiment, the Cas13-IDG composition was delivered as RNA instead of DNA, since expect an eased translation in preclinical and clinical models due to the transient effect of RNA.

### Example 6: Optimization of an RNA-Based Cas13 Delivery System

To further enhance the knockdown of Cas13-IDG in an RNA based delivery system several modifications were tested by measuring the knockdown efficiency against a co-transfected Nanoluciferase mRNA. First, it was shown that JetMessenger transfection reagent is preferred over MessengerMax. The addition of a part of the 5′ beta globin UTR and two copies of the 3′ beta globin UTR increased the stability of the Cas13 mRNA. Switching from commonly used co-transcriptional ARCA-based 5′ capping to post-transcriptional capping by Vaccina Capping System and 2′-O-Methyltransferase further improved the knockdown efficiency. Replacing UTP in the Cas13 mRNA by N1-Methylpseudo-UTP increased the Cas13 protein expression, which was shown by a Cas13-P2A-mRuby3 fusion construct (FIG. 10B). Besides improving the Cas13 mRNA it was found that stabilizing the gRNA by replacing the two 5′ and 3′ terminal nucleotides by 2′-O-methyl phosphorothioate modified nucleotides further improved the knockdown efficiency (FIG. 10A).

### Example 7: Illustration of Cas13d Variants Fused to Different Localization Signals

Cas13d was fused to different single or tandem localization signals to induce a nuclear/cytosolic shuttling (FIG. 11A). A co-transfected nanoluciferase was targeted by two gRNAs in combination with different localized Cas13 variants. A combination of two NLS, along with one NES signal (Cas13d-NLS-NES/Cas13d-IDG) was found to be optimal, likely due to sufficient nuclear import to pick up the gRNA and cytosolic localization to efficiently target the mainly cytosolic nanoluciferase mRNA (FIG. 11B).

### Example 8: Localization of Cas13d Protein Variants Fused to Different Localization Signals

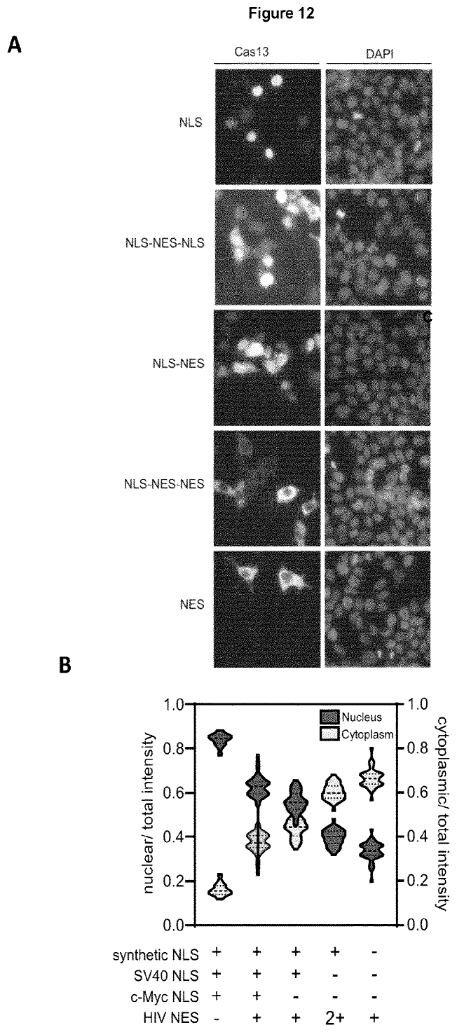

To analyze the impact of different single or tandem localization sequences on the localization of Cas13d immunohistochemistry was performed. Different fusion constructs were transfected and imaged in a high-content analysis system (FIG. 12A). Subsequently, fluorescence intensity measurements for cytosolic and nuclear fractions were performed for 100 cells for each construct. Analysis of cytosolic to nuclear distribution revealed a gradual shift of the protein localization to the cytosol for decreasing NLS and increasing NES signals (FIG. 12B). Cas13d-NES-NLS shows an intermediate localization, supporting the hypothesized shuttling mechanism.

### Example 9: Stability of Cas13d Variants Fused to Different Localization Signals

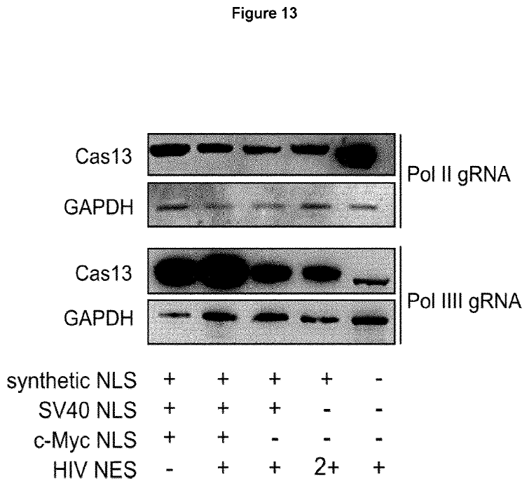

To test if the gRNA and related protein localization has an impact on the Cas13 stability, Western Blot analysis were performed. Therefore Cas13 variants, fused to different localization signals were transfected in HEK293T cells and subsequently analyzed. A polymerase II driven gRNA is exported into the cytosol and therefore stabilizes a mainly cytosolic Cas13 variant, while a polymerase III driven gRNA remains in the nucleus and therefore stabilized preferable a nuclear Cas13 variant (FIG. 13).

### Example 10: Comparison of Knockdown Efficiencies Against Venezuelan Equine Encephalitis (VEE) Replicon

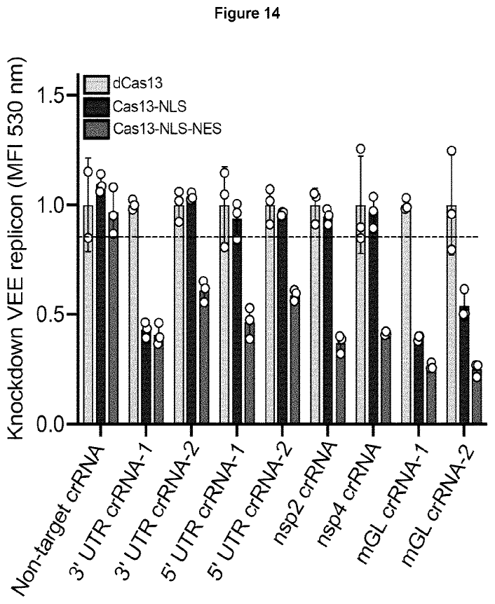

To study the impact of partially cytosolic Cas13d-NLS-NES on the knockdown of cytosolic RNAs, a self-replicating RNA, derived from Venezuelan Equine Encephalitis (VEE) virus, was targeted to mimic a viral infection. VEE is replicating exclusively in the cytosol, without passing the nuclear barrier where conventual Cas13d-NLS is located. A mGreenLantern reporter gene was cloned on a VEE replicon expression construct to measure the replication rate of VEE replicon. The reporter replicon was in vitro transcribed and transfected into HEK293T cells. After 2 weeks of selection, different Cas13 constructs were transfected and their impact on VEE replication was analyzed via flow cytometry. For all gRNAs tested, Cas13d-NLS-NES outperforms Cas13d-NLS and therefore supports that Cas13d-NLS-NES is more efficient in degrading cytosolic RNAs compared to previously described Cas13-NLS (FIG. 14).

### Example 11: Continuous Analysis of SARS-CoV-2-GFP Replication For Treatment Conditions With Cas13d-IDG and Different gRNAs

SARS-CoV-2 is a plus-strand RNA virus, which replicates exclusively in the cytosol, while Cas13d-NLS-NES efficiently degrades cytosolic RNA. To measure the impact of Cas13-NLS-NES on viral replication, HEK293T-ACE2 cells were transfected with Cas13d-NLS-NES coding mRNA along with different gRNAs and infected with SARS-CoV-2-GFP. SARS-CoV-2-GFP encodes an additional green fluorescent protein on the viral genome, enabling the measurement of viral replication in a time course experiment. A chemically modified gRNA, targeting the viral 3′UTR and a pool of gRNAs, targeting the 5′ and 3′ viral UTR strongly inhibited viral replication (FIG. 15A). To further validate these results, different SARS-CoV-2 variants (alpha and delta) were targeted by transfecting Cas13d-NLS-NES along with a chemically modified gRNA (FIG. 15B). The viral load of SARS-CoV-2 was subsequently measured by RT-qPCR for either the viral N or RdRp gene, confirming that the established system is able to inhibit SARS-CoV-2 variants without further modifications.

### Example 12: Comparison of Knockdown Efficiencies For Two gRNAs Targeting a Co-Transfected Nanoluciferase For NES or Tandem NLS-NES Fused to Cas13

Cas13d-NLS-NES travels to the nucleus to pick up the gRNA while CTE fused gRNAs are exported to the cytosol by itself. The combination of both export systems could have an additive effect. Therefore, HEK293T cells were transfected with two gRNAs targeting nanoluciferase, either with or without fused CTE motif and either Cas13-NES or Cas13-NLS-NES. 48 h after the transfection, knockdown efficiencies were measured. Both gRNA export strategies alone performed similar, but the synergistic effect of both export systems further maximized the efficiency (FIG. 16).

### Example 13: Illustration of Different crRNA Export Strategies

Conventual gRNAs are expressed by polymerase III promoters (e.g. U6 promoter) and therefore remain in the nucleus of expressing cells. To target cytoplasmic RNAs, different gRNA export strategies were exploited. Polymerase II driven gRNAs (e.g. CAG promoter) are capped, polyadenylated and therefore exported. Alternatively, viral RNA export elements, such as CTE or VARdm drive the export of gRNAs from the nucleus to the cytosol (FIG. 17A). These export strategies were applied to two gRNAs, targeting a co-transfected nanoluciferase and compared for either a cytosolic or nuclear Cas13 protein (FIG. 17B). As previously shown, conventual pol III driven gRNAs are more efficient in combination with a nuclear Cas13 system. Pol II gRNA expression and fusing viral export motifs favor a cytosolic over a nuclear Cas13 protein. gRNAs fused to the viral CTE motif are most efficient in driving cytosolic knockdown by Cas13d-NES.

The invention is further characterized by the following items:

### Items

1. A composition comprising a clustered, regularly interspaced, short palindromic repeats (CRISPR) system comprising i) at least one CRISPR-associated protein 13 (Cas13) fused with at least one nuclear localization signal (NLS) fused to at least one nuclear export sequence (NES) and ii) at least one guide RNA (gRNA) capable of hybridizing with one or more target RNA molecules.
2. The composition of item 1, wherein said Cas13 protein is encoded by a nucleotide sequence.
3. The composition of item 1 or 2, wherein said gRNA is encoded by a nucleotide sequence.
4. The composition of any one of the preceding items, wherein said Cas13 protein is fused with said at least one NLS fused to said at least one NES via a linker.
5. The composition of any one of the preceding items, wherein said Cas13 protein is a Cas13d protein.
6. The composition of item 5, wherein said Cas13d protein is derived from the genus of Ruminococcus, preferably from Ruminococcus flavevaciens.
7. The composition of any one of the preceding items, wherein said gRNA has a length of at least about 23 nucleotides.
8. The composition of any one of the preceding items, wherein said gRNA has a length of between about 26 to about 30 nucleotides.
9. The composition of any one of the preceding items, wherein said gRNA has at least about 80% complementary sequence identity to said one or more target RNA molecules.
10. The composition of any one of the preceding items, wherein said gRNA is capable of hybridizing to (a) 5′- and/or 3′-untranslated region(s) of said one or more target RNA molecules.
11. The composition of any one of items 3-10, wherein said nucleotide sequence encoding said gRNA is fused with a tRNA or a ribozyme.
12. The composition of any one of the preceding items, wherein said one or more target RNA molecules are viral or bacterial target RNA molecules.
13. The composition of any one of the preceding items, wherein said composition is for inactivating bacterial or viral ssRNA.
14. The composition of any one of the preceding items, wherein said composition is a pharmaceutical composition.
15. The composition of item 14, further comprising at least one pharmaceutical acceptable carrier.
16. The composition of any one of items 1-15 for use in therapy.
17. The composition of any one of items 1-15 for use in a method of preventing or treating a viral or a bacterial disease in a subject, the method comprising administering to the subject a therapeutically effective amount of the composition of any one of items 1-15.
18. The composition for the use of item 17, wherein the viral disease is caused by a RNA virus.
19. The composition for the use of item 18, wherein the viral disease is any one of a coronavirus disease, influenza A, ebola, measles, hepatitis C, tick-borne encephalitis (TBE), dengue fever, yellow fever, or zika fever.
20. The composition for the use of item 19, wherein the viral disease is the COVID-19 disease.
21. A nucleic acid molecule comprising a nucleotide sequence encoding said Cas13 protein and/or said gRNA as defined in any one of the preceding items.
22. A vector comprising the nucleic acid molecule of item 21.
23. A host cell comprising the vector of item 22 or the nucleic acid molecule of item 21.
24. A kit comprising the composition of any one of items 1-15.
25. The kit of item 24, further comprising a delivery system and/or a label.
26. A method of producing the composition of any one of items 1-15, comprising
a) nucleic acid coding for said Cas13 protein and/or said gRNA as defined in any one of items 1-15 by means of genetic engineering methods, thereby producing said composition; optionally
b) obtaining said produced composition of step a).

1. A composition comprising a clustered, regularly interspaced, short palindromic repeats (CRISPR) system comprising i) at least one CRISPR-associated protein 13 (Cas13) or a nucleotide sequence encoding said Cas13 protein fused with at least one nuclear localization signal (NLS) fused to at least one nuclear export sequence (NES) and ii) at least one guide RNA (gRNA) or a nucleotide sequence encoding said gRNA capable of hybridizing with one or more target RNA molecules.

2. The composition of claim 1, wherein said Cas13 protein is fused with said at least one NLS fused to said at least one NES via a linker.

3. The composition of claim 1 or 2, wherein the Cas13 protein is fused with two NLS fused to one NES.

4. The composition of claim 3, wherein the two NLS comprise the SV40 NLS having the amino acid sequence as depicted in SEQ ID NO: 5 and the NLS consensus sequence having the amino acid sequence as depicted in SEQ ID NO: 3 and the one NES comprises the HIV NES having the amino acid sequence as depicted in SEQ ID NO: 4.

5. The composition of any one of the preceding claims, wherein said Cas13 protein is a Cas13d protein.

6. The composition of claim 5, wherein said Cas13d protein is derived from the genus of Ruminococcus, preferably from Ruminococcus flavevaciens.

7. The composition of any one of the preceding claims, wherein said gRNA has a length of at least about 23 nucleotides.

8. The composition of any one of the preceding claims, wherein said gRNA has a length of between about 26 to about 30 nucleotides.

9. The composition of any one of the preceding claims, wherein said gRNA has at least about 80% complementary sequence identity to said one or more target RNA molecules.

10. The composition of any one of the preceding claims, wherein said gRNA is capable of hybridizing to (a) 5′- and/or 3′-untranslated region(s) of said one or more target RNA molecules.

11. The composition of any one of claims 1-10, wherein said nucleotide sequence encoding said gRNA is fused with a tRNA or a ribozyme.

12. The composition of any one of claims 1-11, wherein said nucleotide sequence encoding said Cas13 protein and/or said gRNA comprises any one of the following modifications: modified 5′ and/or 3′ UTRs, post- or cotranscriptional addition of a 5′ CAP structure, replacement of UTP by N1-methylpseudo-UTP, or replacement of the two 5′ and/or 3′ terminal nucleotides by 2′-O-methyl-3′P-thioate.

13. The composition of any one of claims 1-12, wherein said nucleotide sequence encoding said gRNA is fused with at least one viral export element.

14. The composition of claim 13, wherein said at least one viral export element is a constitutive transport element (CTE) or adenovirus VA1 RNA (VARdm).

15. The composition of any one of the preceding claims, wherein said one or more target RNA molecules are viral or bacterial target RNA molecules.

16. The composition of any one of the preceding claims, wherein said composition is for inactivating bacterial or viral ssRNA.

17. The composition of any one of the preceding claims, wherein said composition is a pharmaceutical composition.

18. The composition of claim 17, further comprising at least one pharmaceutical acceptable carrier.

19. The composition of any one of claims 1-18 for use in therapy.

20. The composition of any one of claims 1-18 for use in a method of preventing or treating a viral or a bacterial disease in a subject, the method comprising administering to the subject a therapeutically effective amount of the composition of any one of claims 1-18.

21. The composition for the use of claim 20, wherein the viral disease is caused by a RNA virus.

22. The composition for the use of claim 21, wherein the viral disease is any one of a coronavirus disease, influenza A, ebola, measles, hepatitis C, tick-borne encephalitis (TBE), Venezuelan Equine Encephalitis (VEE) viral infection, dengue fever, yellow fever, or zika fever.

23. The composition for the use of claim 22, wherein the viral disease is the COVID-19 disease.

24. A nucleic acid molecule comprising a nucleotide sequence encoding said Cas13 protein and said gRNA as defined in any one of claims 1-18.

25. A vector comprising the nucleic acid molecule of claim 24.

26. A host cell comprising the vector of claim 25 or the nucleic acid molecule of claim 24.

27. A kit comprising the composition of any one of claims 1-18.

28. The kit of claim 27, further comprising a delivery system and/or a label.

29. A method of producing the composition of any one of claims 1-18, comprising
a) nucleic acid coding for said Cas13 protein and said gRNA as defined in any one of claims 1-18 by means of genetic engineering methods, thereby producing said composition; optionally
b) obtaining said produced composition of step a).

30. A composition comprising a clustered, regularly interspaced, short palindromic repeats (CRISPR) system comprising i) at least one CRISPR-associated protein 13 (Cas13) or a nucleotide sequence encoding said Cas13 protein fused with at least one nuclear localization signal (NLS) or with at least one nuclear export sequence (NES) and ii) at least one guide RNA (gRNA) or a nucleotide sequence encoding said gRNA capable of hybridizing with one or more target RNA molecules, which is fused to at least one viral export element.

31. The composition of claim 30, wherein said at least one viral export element is a constitutive transport element (CTE) or adenovirus VA1 RNA (VARdm).

32. The composition of claim 30 or 31, wherein said Cas13 protein is a Cas13d protein.

33. The composition of claim 32, wherein said Cas13d protein is derived from the genus of Ruminococcus, preferably from Ruminococcus flavevaciens.

34. The composition of claims 30-33, wherein said gRNA has a length of at least about 23 nucleotides.

35. The composition of claims 30-34, wherein said gRNA has a length of between about 26 to about 30 nucleotides.

36. The composition of claims 30-35, wherein said gRNA has at least about 80% complementary sequence identity to said one or more target RNA molecules.

37. The composition of claims 30-36, wherein said gRNA is capable of hybridizing to (a) 5′- and/or 3′-untranslated region(s) of said one or more target RNA molecules.

38. The composition of claims 30-37, wherein said nucleotide sequence encoding said gRNA is fused with a tRNA or a ribozyme.

39. The composition of claims 30-38, wherein said one or more target RNA molecules are viral or bacterial target RNA molecules.

40. The composition of claims 30-39, wherein said composition is for inactivating bacterial or viral ssRNA.

41. The composition of claims 30-40, wherein said composition is a pharmaceutical composition.

42. The composition of claim 41, further comprising at least one pharmaceutical acceptable carrier.

43. The composition of any one of claims 30-42 for use in therapy.

44. The composition of any one of claims 30-42 for use in a method of preventing or treating a viral or a bacterial disease in a subject, the method comprising administering to the subject a therapeutically effective amount of the composition of any one of claims 30-42.

45. The composition for the use of claim 44, wherein the viral disease is caused by a RNA virus.

46. The composition for the use of claim 45, wherein the viral disease is any one of a coronavirus disease, influenza A, ebola, measles, hepatitis C, tick-borne encephalitis (TBE), Venezuelan Equine Encephalitis (VEE) viral infection, dengue fever, yellow fever, or zika fever.

47. The composition for the use of claim 46, wherein the viral disease is the COVID-19 disease.

48. A nucleic acid molecule comprising a nucleotide sequence encoding said Cas13 protein and said gRNA as defined in any one of claims 30-42.

49. A vector comprising the nucleic acid molecule of claim 48.

50. A host cell comprising the vector of claim 49 or the nucleic acid molecule of claim 48.

51. A kit comprising the composition of any one of claims 30-42.

52. The kit of claim 51, further comprising a delivery system and/or a label.

53. A method of producing the composition of any one of claims 30-42, comprising
a) nucleic acid coding for said Cas13 protein and said gRNA as defined in any one of claims 30-42 by means of genetic engineering methods, thereby producing said composition; optionally
b) obtaining said produced composition of step a).
